SEQUENCE LISTING (42 sequences)

SEQ ID NO: 1 - DNA, 2021 bp, Arabidopsis thaliania
atcaaaaaaaaagtttcaatttttgaaagctctgagaaatgaatctatcattctctctctctatctctatcttccttttcagatttcgcttcttcaattcatgaaatcctcgtgattctactttaatgcttctctttttttacttttccaagtctctgaatattcaaagtatatatcttttgttttcaaacttttgcagaattgtcttcaagcttccaaatttcagttaaaggtctcaactttgcagaattttcctctaaaggttcagactttggggtaaaggtgtcaactttggcgatgggtcttgacggaaacaatggtggaggggtttggttaaacggtggtggtggagaaagggaagagaacgaggaaggttcatggggaaggaatcaagaagatggttcttctcagtttaagcctatgcttgaaggtgattggtttagtagtaaccaaccacatccacaagatcttcagatgttacagaatcagccagatttcagatactttggtggttttccttttaaccctaatgataatcttcttcttcaacactctattgattcttcttcttcttgttctccttctcaagcttttagtcttgacccttctcagcaaaatcagttcttgtcaactaacaacaacaagggttgtcttctcaatgttccttcttctgcaaacccttttgataatgcttttgagtttggctctgaatctggttttcttaaccaaatccatgctcctatttcgatggggtttggttctttgacacaattggggaacagggatttgagttctgttcctgatttcttgtctgctcggtcacttcttgcgccggaaagcaacaacaacaacacaatgttgtgtggtggtttcacagctccgttggagttggaaggttttggtagtcctgctaatggtggttttgttgggaacagagcgaaagttctgaagcctttagaggtgttagcatcgtctggtgcacagcctactctgttccagaaacgtgcagctatgcgtcagagctctggaagcaaaatgggaaattcggagagttcgggaatgaggaggtttagtgatgatggagatatggatgagactgggattgaggtttctgggttgaactatgagtctgatgagataaatgagagcggtaaagcggctgagagtgttcagattggaggaggaggaaagggtaagaagaaaggtatgcctgctaagaatctgatggctgagaggagaaggaggaagaagcttaatgataggctttatatgcttagatcagttgtccccaagatcagcaaaatggatagagcatcaatacttggagatgcaattgattatctgaaggaacttctacaaaggatcaatgatcttcacaatgaacttgagtcaactcctcctggatctttgcctccaacttcatcaagcttccatccgttgacacctacaccgcaaactctttcttgtcgtgtcaaggaagagttgtgtccctcttctttaccaagtcctaaaggccagcaagctagagttgaggttagattaagggaaggaagagcagtgaacattcatatgttctgtggtcgtagaccgggtctgttgctcgctaccatgaaagctttggataatcttggattggatgttcagcaagctgtgatcagctgttttaatgggtttgccttggatgttttccgcgctgagcaatgccaagaaggacaagagatactgcctgatcaaatcaaagcagtgcttttcgatacagcagggtatgctggtatgatctgatctgatcctgacttcgagtccattaagcatctgttgaagcagagctagaagaactaagtccctttaaatctgcaattttcttctcaactttttttcttatgtcataacttcaatctaagcatgtaatgcaattgcaaatgagagttgtttttaaattaagcttttgagaacttgaggttgttgttgttggatacataacttcaaccttttattagcaatgttaacttccatttatgtct

SEQ ID NO: 2 - protein, 494 aa, Arabidopsis thaliania
MGLDGNNGGGVWLNGGGGEREENEEGSWGRNQEDGSSQFKPMLEGDWFSSNQPHPQDLQMLQNQPDFRYFGGFPFNPNDNLLLQHSIDSSSSCSPSQAFSLDPSQQNQFLSTNNNKGCLLNVPSSANPFDNAFEFGSESGFLNQIHAPISMGFGSLTQLGNRDLSSVPDFLSARSLLAPESNNNNTMLCGGFTAPLELEGFGSPANGGFVGNRAKVLKPLEVLASSGAQPTLFQKRAAMRQSSGSKMGNSESSGMRRFSDDGDMDETGIEVSGLNYESDEINESGKAAESVQIGGGGKGKKKGMPAKNLMAERRRRKKLNDRLYMLRSVVPKISKMDRASILGDAIDYLKELLQRINDLHNELESTPPGSLPPTSSSFHPLTPTPQTLSCRVKEELCPSSLPSPKGQQARVEVRLREGRAVNIHMFCGRRPGLLLATMKALDNLGLDVQQAVISCFNGFALDVFRAEQCQEGQEILPDQIKAVLFDTAGYAGMI

SEQ ID NO: 3 - protein, 828 aa, Arabidopsis thaliania
MESREDSFISKEKKSTMKKEKQAIASQRNRRRVIKNRGNGKRLIASLSQRKRRRIPRGRGNEKAVFAPSSLPNDVVEEIFLRLPVKAIIQLKSLSKQWRSTIESRSFEERHLKIVERSRVDFPQVMVMSEEYSLKGSKGNQPRPDTDIGFSTICLESASILSSTLITFPQGFQHRIYASESCDGLFCIHSLKTQAIYVVNPATRWFRQLPPARFQILMQKLYPTQDTWIDIKPVVCYTAFVKANDYKLVWLYNSDASNPNLGVTKCEVFDFRANAWRYLTCTPSYRIFPDQVPAATNGSIYWFTEPYNGEIKVVALDIHTETFRVLPKINPAIASSDPDHIDMCTLDNGLCMSKRESDTLVQEIWRLKSSEDSWEKFDMNSDGVWLDGSGESPEVNNGEAASWVRNPDEDWFNNPPPPQHTNQNDFRFNGGFPLNPSENLLLLLQQSIDSSSSSSPLLHPFTLDAASQQQQQQQQQQEQSFLATKACIVSLLNVPTINNNTFDDFGFDSGFLGQQFHGNHQSPNSMNFTGLNHSVPDFLPAPENSSGSCGLSPLFSNRAKVLKPLQVMASSGSQPTLFQKRAAMRQSSSSKMCNSESSSEMRKSSYEREIDDTSTGIIDISGLNYESDDHNTNNNKGKKKGMPAKNLMAERRRRKKLNDRLYMLRSVVPKISKMDRASILGDAIDYLKELLQRINDLHTELESTPPSSSSLHPLTPTPQTLSYRVKEELCPSSSLPSPKGQQPRVEVRLREGKAVNIHMFCGRRPGLLLSTMRALDNLGLDVQQAVISCFNGFALDVFRAEQCQEDHDVLPEQIKAVLLDTAGYAGLV

SEQ ID NO: 4 - protein, 351 aa, Arabidopsis thaliania
MELSTQMNVFEELLVPTKQETTDNNINNLSFNGGFDHHHHQFFPNGYNIDYLCFNNEEEDENTLLYPSSFMDLISQPPPLLLHQPPPLQPLSPPLSSSATAGATFDYPFLEALQEIIDSSSSSPPLILQNGQEENFNNPMSYPSPLMESDQSKSFSVGYCGGETNKKKSKKLEGQPSKNLMAERRRRKRLNDRLSMLRSIVPKISKMDRTSILGDAIDYMKELLDKINKLQDEEQELGNSNNSHHSKLFGDLKDLNANEPLVRNSPKFEIDRRDEDTRVDICCSPKPGLLLSTVNTLETLGLEIEQCVISCFSDFSLQASCSEGAEQRDFITSEDIKQALFRNAGYGGSCL

SEQ ID NO: 5 - protein, 315 aa, Arabidopsis thaliania
METELTQLRKQESNNLNGVNGGFMAIDQFVPNDWNFDYLCFNNLLQEDDNIDHPSSSSLMNLISQPPPLLHQPPQPSSPLYDSPPLSSAFDYPFLEDIIHSSYSPPPLILPASQENTNNYSPLMEESKSFISIGETNKKRSNKKLEGQPSKNLMAERRRRKRLNDRLSLLRSIVPKITKMDRTSILGDAIDYMKELLDKINKLQEDEQELGSNSHLSTLITNESMVRNSLKFEVDQREVNTHIDICCPTKPGLVVSTVSTLETLGLEIEQCVISCFSDFSLQASCFEVGEQRYMVTSEATKQALIRNAGYGGRCL

SEQ ID NO: 6 - protein, 623 aa, Arabidopsis thaliania
MTDYRLQPTMNLWTTDDNASMMEAFMSSSDISTLWPPASTTTTTATTETTPTPAMEIPAQAGFNQETLQQRLQALIEGTHEGWTYAIFWQPSYDFSGASVLGWGDGYYKGEEDKANPRRRSSSPPFSTPADQEYRKKVLRELNSLISGGVAPSDDAVDEEVTDTEWFFLVSMTQSFACGAGLAGKAFATGNAVWVSGSDQLSGSGCERAKQGGVFGMHTIACIPSANGVVEVGSTEPIRQSSDLINKVRILFNFDGGAGDLSGLNWNLDPDQGENDPSMWINDPIGTPGSNEPGNGAPSSSSQLFSKSIQFENGSSSTITENPNLDPTPSPVHSQTQNPKFNNTFSRELNFSTSSSTLVKPRSGEILNFGDEGKRSSGNPDPSSYSGQTQFENKRKRSMVLNEDKVLSFGDKTAGESDHSDLEASVVKEVAVEKRPKKRGRKPANGREEPLNHVEAERQRREKLNQRFYALRAVVPNVSKMDKASLLGDAIAYINELKSKVVKTESEKLQIKNQLEEVKLELAGRKASASGGDMSSSCSSIKPVGMEIEVKIIGWDAMIRVESSKRNHPAARLMSALMDLELEVNHASMSVVNDLMIQQATVKMGFRIYTQEQLRASLISKIG

SEQ ID NO: 7 - protein, 592 aa, Arabidopsis thaliania
MNGTTSSINFLTSDDDASAAAMEAFIGTNHHSSLFPPPPQQPPQPQFNEDTLQQRLQALIESAGENWTYAIFWQISHDFDSSTGDNTVILGWGDGYYKGEEDKEKKKNNTNTAEQEHRKRVIRELNSLISGGIGVSDESNDEEVTDTEWFFLVSMTQSFVNGVGLPGESFLNSRVIWLSGSGALTGSGCERAGQGQIYGLKTMVCIATQNGVVELGSSEVISQSSDLMHKVNNLFNFNNGGGNNGVEASSWGFNLNPDQGENDPALWISEPTNTGIESPARVNNGNNSNSNSKSDSHQISKLEKNDISSVENQNRQSSCLVEKDLTFQGGLLKSNETLSFCGNESSKKRTSVSKGSNNDEGMLSFSTVVRSAANDSDHSDLEASVVKEAIVVEPPEKKPRKRGRKPANGREEPLNHVEAERQRREKLNQRFYSLRAVVPNVSKMDKASLLGDAISYINELKSKLQQAESDKEEIQKKLDGMSKEGNNGKGCGSRAKERKSSNQDSTASSIEMEIDVKIIGWDVMIRVQCGKKDHPGARFMEALKELDLEVNHASLSVVNDLMIQQATVKMGSQFFNHDQLKVALMTKVGENY

SEQ ID NO: 8 - protein, 610 aa, Zea mays
MALSASRVQQAEELLQRPAERQLMRSQLAAAARSINWSYALFWSISDTQPGVLTWTDGFYNGEVKTRKISNSVELTSDQLVMQRSDQLRELYEALLSGEGDRRAAPARPAGSLSPEDLGDTEWYYVVSMTYAFRPGQGLPGRSFASDEHVWLCNAHLAGSKAFPRALLAKSASIQSILCIPVMGGVLELGTTDTVPEAPDLVSRATAAFWEPQCPSSSPSGRANETGEAAADDGTFAFEELDHNNGMDDIEAMTAAGGHGQEEELRLREAEALSDDASLEHITKEIEEFYSLCDEMDLQALPLPLEDGWTVDASNFEVPCSSPQPAPPPVDRATANVAADASRAPVYGSRATSFMAWTRSSQQSSCSDDAAPAAVVPAIEEPQRLLKKVVAGGGAWESCGGATGAAQEMSGTGTKNHVMSERKRREKLNEMFLVLKSLLPSIHRVNKASILAETIAYLKELQRRVQELESSREPASRPSETTTRLITRPSRGNNESVRKEVCAGSKRKSPELGRDDVERPPVLTMDAGTSNVTVTVSDKDVLLEVQCRWEELLMTRVFDAIKSLHLDVLSVQASAPDGFMGLKIRAQFAGSGAVVPWMISEALRKAIGKR

SEQ ID NO: 9 - protein, 379 aa, Arabidopsis thaliania
MDESSIIPAEKVAGAEKKELQGLLKTAVQSVDWTYSVFWQFCPQQRVLVWGNGYYNGAIKTRKTTQPAEVTAEEAALERSQQLRELYETLLAGESTSEARACTALSPEDLTETEWFYLMCVSFSFPPPSGMPGKAYARRKHVWLSGANEVDSKTFSRAILAKTVVCIPMLDGVVELGTTKKNGKEHQQVKTAPSSQWVLKQMIFRVPFLHDNTKDKRLPREDLSHVVAERRRREKLNEKFITLRSMVPFVTKMDKVSILGDTIAYVNHLRKRVHELENTHHEQQHKRTRTCKRKTSEEVEVSIIENDVLLEMRCEYRDGLLLDILQVLHELGIETTAVHTSVNDHDFEAEIRAKVRGKKASIAEVKRAIHQVIIHDTNL

SEQ ID NO: 10 - protein, 432 aa, Arabidopsis thaliania
MEQVFADWNFEDNFHMSTNKRSIRPEDELVELLWRDGQVVLQSQARREPSVQVQTHKQETNQETVQKPNYAALDDQETVSWIQYPPDDVIDPFESEFSSHFFSSIDHLGGPEKPRMIEETVKHEAQAMAPPKFRSSVITVGPSHCGSNQSTNIHQATTLPVSMSDRSKNVEERLDTSSGGSSGCSYGRNNKETVSGTSVTIDRKRKHVMDADQESVSQSDIGLTSTDDQTMGNKSSQRSGSTRRSRAAEVHNLSERRRRDRINERMKALQELIPHCSRTDKASILDEAIDYLKSLQMQLQVMWMGSGMAAAAAAAASPMMFPGVQSSPYINQMAMQSQMQLSQFPVMNRSAPQNHPGLVCLNPVQLQLQAQNQILSEQLARYMGGIPQMPPAGNQTVQQQPADMLGFGSPAGPQSQLSAPATTDSLHMGKIG

SEQ ID NO: 11 - protein, 430 aa, Arabidopsis thaliania
MEHQGWSFEENYSLSTNRRSIRPQDELVELLWRDGQVVLQSQTHREQTQTQKQDHHEEALRSSTFLEDQETVSWIQYPPDEDPFEPDDFSSHFFSTMDPLQRPTSETVKPKSSPEPPQVMVKPKACPDPPPQVMPPPKFRLTNSSSGIRETEMEQYSVTTVGPSHCGSNPSQNDLDVSMSHDRSKNIEEKLNPNASSSSGGSSGCSFGKDIKEMASGRCITTDRKRKRINHTDESVSLSDAIGNKSNQRSGSNRRSRAAEVHNLSERRRRDRINERMKALQELIPHCSKTDKASILDEAIDYLKSLQLQLQVMWMGSGMAAAAASAPMMFPGVQPQQFIRQIQSPVQLPRFPVMDQSAIQNNPGLVCQNPVQNQIISDRFARYIGGFPHMQAATQMQPMEMLRFSSPAGQQSQQPSSVPTKTTDGSRLDH

SEQ ID NO: 12 - protein, 165 aa, Danio rerio
MSDNDDIEVDSDADSPRFHGVADKRAHHNALERKRRDHIKDSFHSLRDSVPALQGEKQSIKQASRAQILDKATEYIQYMRRKNHTHQQDIDDLKRQNALLEQQVRALEKVKGTTQLQANYSSSDSSLYTNPKGQAVSAFDGGSDSSSGSEPEEQRTRKKHRPEDS

SEQ ID NO: 13 - protein, 440 aa, Homo sapiens [sequence omitted]

SEQ ID NO: 14 - DNA, 32 bp, Artificial Sequence
  Synthetic DNA
tcatggatccaccatttgttaatgcatgatgg

SEQ ID NO: 15 - DNA, 26 bp, Artificial Sequence
  Synthetic DNA
gctcaagctttctgttctagttcagg

SEQ ID NO: 16 - DNA, 24 bp, Artificial Sequence
  Synthetic DNA
ttcgatttttatttccatttttgg

SEQ ID NO: 17 - DNA, 22 bp, Artificial Sequence
  Synthetic DNA
ccaaacgtccttgagtcttgat

SEQ ID NO: 18 - DNA, 26 bp, Artificial Sequence
  Synthetic DNA
taaaactcagattattatttccattt

SEQ ID NO: 19 - DNA, 20 bp, Artificial Sequence
  Synthetic DNA
gaggagccacgtagagggcc

SEQ ID NO: 20 - DNA, 24 bp, Artificial Sequence
  Synthetic DNA
cgtggatcacagcaatacagagcc

SEQ ID NO: 21 - DNA, 25 bp, Artificial Sequence
  Synthetic DNA
cctcctgcacttccacttcgtcttc

SEQ ID NO: 22 - DNA, 37 bp, Artificial Sequence
  Synthetic DNA
agggatccggaccaccgtcaataacatcgttaagtag

SEQ ID NO: 23 - DNA, 40 bp, Artificial Sequence
  Synthetic DNA
cgaattctaaccgccattaactatgtctcctctctatctc

SEQ ID NO: 24 - DNA, 37 bp, Artificial Sequence; identical to SEQ ID NO: 22
  Synthetic DNA

SEQ ID NO: 25 - DNA, 34 bp, Artificial Sequence
  Synthetic DNA
cgaattcgccaaagttgacacctttaccccaaag

SEQ ID NO: 26 - DNA, 28 bp, Artificial Sequence
  Synthetic DNA
gcgatgggtcttgacggaaacaatggtg

SEQ ID NO: 27 - DNA, 31 bp, Artificial Sequence
  Synthetic DNA
tcagatcataccagcataccctgctgtatcg

SEQ ID NO: 28 - DNA, 21 bp, Artificial Sequence
  Synthetic DNA
gtcaagaggttctcagcagta

SEQ ID NO: 29 - DNA, 21 bp, Artificial Sequence
  Synthetic DNA
tcaccttcttgatccgcagtt

SEQ ID NO: 30 - DNA, 36 bp, Artificial Sequence
  Synthetic DNA
gctctagagcgatgggtcttgacggaaacaatggtg

SEQ ID NO: 31 - DNA, 39 bp, Artificial Sequence
  Synthetic DNA
ggggtacctcagatcataccagcataccctgctgtatcg

SEQ ID NO: 32 - DNA, 36 bp, Artificial Sequence
  Synthetic DNA
aggaattcgcgatgggtcttgacggaaacaatggtg

SEQ ID NO: 33 - DNA, 39 bp, Artificial Sequence
  Synthetic DNA
ctggatcctcagatcataccagcataccctgctgtatcg

SEQ ID NO: 34 - DNA, 20 bp, Artificial Sequence
  Synthetic DNA
accccaccatttgttaatgc

SEQ ID NO: 35 - DNA, 20 bp, Artificial Sequence
  Synthetic DNA
acaattacaactgcatgctt

SEQ ID NO: 36 - DNA, 20 bp, Artificial Sequence
  Synthetic DNA
aatgttacatttgatcattc

SEQ ID NO: 37 - DNA, 20 bp, Artificial Sequence
  Synthetic DNA
ctctggacacatggcagatc

SEQ ID NO: 38 - DNA, 20 bp, Artificial Sequence
  Synthetic DNA
cattttacaattgcttcgct

SEQ ID NO: 39 - DNA, 20 bp, Artificial Sequence
  Synthetic DNA
atataattaactacttttat

SEQ ID NO: 40 - DNA, 20 bp, Artificial Sequence
  Synthetic DNA
gactcgtttcgcgatccgat

SEQ ID NO: 41 - DNA, 20 bp, Artificial Sequence
  Synthetic DNA
ctctggacacatggcagatc

SEQ ID NO: 42 - DNA, 20 bp, Artificial Sequence
  Synthetic DNA
ctctggaaccagtgcagatc

(19) United States
(12) Patent Application Publication
(10) Pub. No.: US 2003/0233681 A1
(43) Pub. Date: Dec. 18, 2003

(54) ICE1, a regulator of cold induced transcriptome and freezing tolerance in plants

(75) Inventors: Jian-Kang Zhu, Tucson, AZ (US); Viswanathan Chinnusamy, New Delhi (IN); Masaru Ohta, Ibaraki (JP); Siddhartha Kanrar, Tucson, AZ (US); Byeong-ha Lee, Tucson, AZ (US); Manu Agarwal, Tucson, AZ (US)

Correspondence Address: OBLON, SPIVAK, MCCLELLAND, MAIER & NEUSTADT, P.C., 1940 DUKE STREET, ALEXANDRIA, VA, 22314, US

(73) Assignee: Arizona Bd of Regents/Behalf of Univ. of Arizona, Tucson, AZ

(21) Appl. No.: 10/425,913

(22) Filed: Apr. 30, 2003

Related U.S. Application Data
(60) Provisional application No. 60/377,897, filed on May 2, 2002.
(60) Provisional application No. 60/377,469, filed on May 1, 2002.

(51) Int. Cl. A01H 1/00; C12N 15/82; C12N 5/04; C12Q 1/68; C07H 21/04

(52) U.S. Cl. 800/289; 435/6; 435/69.1; 435/320.1; 435/419; 536/23.6

(57) ABSTRACT

The present invention provides methods and compositions for improving cold acclimation of plants. More specifically, the present invention utilizes overexpression of ICE1 in plants and plant cells.

### CROSS-REFERENCE TO RELATED APPLICATIONS

This application claims benefit to U.S. Provisional Application Serial No. 60/377,469, filed on May 1, 2002, and U.S. Provisional Application Serial No. 60/377,897, filed on May 2, 2002, both or which are incorporated herein by reference in their entirety.

### STATEMENT REGARDING FEDERALLY SPONSORED RESEARCH

This work was supported by the National Science Foundation Grant No. IBN9808398 and by the U.S. Department of Agriculture USDA/CSREES Grant No. 00-35100-9426. The United States government is entitled to certain rights in the present application.

### BACKGROUND OF THE INVENTION

1. Field of the Invention

The present invention relates to proteins and nucleic acids related to regulation of cold induced transcriptome and freezing tolerance in plants.

2. Discussion of the Background

Cold is an environmental factor that limits the geographical distribution and growing season of many plant species, and it often adversely affects crop quality and productivity (Thomashow 1999). For example, most temperate plants can acquire tolerance to freezing temperatures by a process known as cold acclimation in which tolerance arises through a prior exposure to low non-freezing temperatures (Guy 1990; Hughes and Dunn 1996; Browse and Xin 2001). However, plants of tropical and sub-tropical origins are incapable of cold acclimation and, as such, are sensitive to chilling temperatures (0-10° C.). Many studies have suggested that cold-regulated gene expression is critical in plants for both chilling tolerance (Gong et al. 2002; Hsieh et al. 2002) and cold acclimation (Thomashow 1999; Knight et al. 1999; Tahtiharju and Palva 2001). Cold-responsive genes encode a diverse array of proteins, such as enzymes involved in respiration and metabolism of carbohydrates, lipids, phenylpropanoids and antioxidants; molecular chaperones, antifreeze proteins; and others with a presumed function in tolerance to the dehydration caused by freezing (Thomashow 1999; Guy 1990; Mohapatra et al. 1989).

Many of the cold and dehydration responsive genes have one or several copies of the DRE/CRT cis-element in their promoters, which has the core sequence, CCGAC (Yamaguchi-Shinozaki and Shinozaki 1994; Stockinger et al. 1997). A family of transcription factors known as CBFs or DREB1s binds to this element and activates transcription of the downstream cold and dehydration-responsive genes (Stockinger et al. 1997; Liu et al. 1998). Interestingly, the CBF/DREB1 genes are themselves induced by low temperatures. This induction is transient and precedes that of the downstream genes with the DRE/CRT cis-element (Thomashow 1999). Therefore, there is a transcriptional cascade leading to the expression of the DRE/CRT class of genes under cold stress. Ectopic expression of CBFs/DREB1s in plants turns on downstream cold-responsive genes even at warm temperatures and confers improved freezing tolerance (Jagglo-Ottosen et al. 1998; Liu et al. 1998).

Since CBF transcripts begin accumulating within 15 min of plants' exposure to cold, Gilmour et al (1998) proposed that there is a transcription factor already present in the cell at normal growth temperature that recognizes the CBF promoters and induces CBF expression upon exposure to cold stress. Gilmour et al (1998) named the unknown activator(s) as “ICE” (Inducer of CBF Expression) protein(s) and hypothesized that upon exposing a plant to cold, modification of either ICE or an associated protein would allow ICE to bind to CBF promoters and activate CBF transcription.

Genetic analysis in Arabidopsis plants expressing the firefly luciferase reporter gene driven by the CRT/DRE element-containing RD29A promoter (Ishitani et al. 1997) has identified several mutants with de-regulated cold-responsive gene expression. The hos1 (high expression of osmotically responsive genes) mutant shows an enhanced cold-induction of CBFs and their downstream cold responsive genes (Ishitani et al. 1998). HOSI encodes a RING finger protein that is present in the cytoplasm at normal growth temperatures but accumulates in the nucleus upon cold treatment. Since many RING-finger proteins are known to serve as ubiquitin E3 ligases, HOS1 has been proposed to function by targeting certain positive regulator(s) of CBFs for ubiquitination and degradation (Lee et al. 2001). The transcription of CBF genes is also under feedback repression by its own gene product or its downstream target gene products. This was revealed by studies on the los1 mutant that is defective in the translational elongation factor 2 gene (Guo et al. 2002). The los1 mutation blocks cold induction of genes with the CRT/DRE element but causes super-induction of the CBF genes. It was shown that protein synthesis in los1 mutant plants is disrupted specifically in the cold. Therefore, cold-induced CBF transcripts cannot be translated to activate downstream genes, and feedback repression cannot occur, leading to super-induction of CBF transcripts (Guo et al. 2002).

Another Arabidopsis mutation, los2, also impairs cold induction of CRT/DRE element-containing genes (Lee et al., 2002). LOS2 encodes a bi-functional enolase that can bind to the promoter of ZAT10, a zinc finger transcriptional repressor. ZAT10 expression is rapidly and transiently induced by cold in the wild type, and this induction is stronger and more sustained in the los2 mutant. Therefore, LOS2 may control the expression of delayed cold response genes via transcriptional repression of ZAT1 (Lee et al. 2002). The Arabidopsis LOS4 locus is involved in the accumulation of CBF transcripts under cold treatment (Gong et al. 2002). los4-1 mutant plants are sensitive to chilling stress, and the chilling sensitivity can be rescued by ectopic expression of CBF3 (Gong et al. 2002). LOS4 encodes a DEAD-box RNA helicase, suggesting that RNA metabolism may be involved in cold responses.

Since environmental factors, such as cold, limits the geographical distribution and growing season of many plant species, and often adversely affects crop quality and productivity, there remains an ongoing critical need to increase cold acclimation in plants, particularly those plants that are advantageously useful as agricultural crops.

### SUMMARY OF THE INVENTION

It is an object of the present invention to provide methods and compositions for increasing cold acclimation in plants.

It is another object of the present invention to provide plants and plant cells, which have increased cold acclimation.

The objects of the present invention, and others, may be accomplished with a method of increasing cold acclimation in a plant, comprising overexpressing ICE1 in the plant.

The objects of the present invention may also be accomplished with a method of increasing cold acclimation in a plant cell, comprising overexpressing ICE1 in the plant cell.

The objects of the present invention may also be accomplished with a plant or a plant cell transformed with a nucleic acid that encodes ICE1.

Thus, the present invention also provides a method of producing such a plant or plant cell, by transforming a plant or plant cell with the nucleic acid that encodes ICE1.

The present invention also provides an isolated and purified ICE1 having the amino acid sequence of SEQ ID NO: 2.

The present invention also provides a method of producing the ICE1 described above, comprising culturing host cells that have been transformed with a nucleic acid encoding ICE1 under conditions in which ICE1 is expressed, and isolating ICE1.

In another embodiment, the present invention provides an isolated and purified enzyme having ICE1 transcriptional activator activity, wherein the amino acid sequence of the enzyme has a homology of from 70% to less than 100% to SEQ ID NO: 2.

The present invention also provides a method of producing the enzyme described above, comprising culturing host cells that have been transformed with a nucleic acid encoding the enzyme under conditions in which the enzyme is expressed, and isolating the enzyme.

The present invention also provides a method of increasing cold acclimation in a plant, comprising overexpressing an ICE1 transcriptional activator in the plant.

The present invention also provides a method of increasing cold acclimation in a plant by increasing the expression of one or more additional transcription factors selected from the group consisting of a CBF transcription factor and a DREB1 transcription factor and/or by increasing expression of one or more cold-responsive genes.

The present invention has been accomplished using a genetic screen (Chinnusamy et al. 2002) to identify cold signaling components upstream of the CBF proteins. A cold-responsive bioluminescent Arabidpsis plant was engineered by expressing the firefly luciferase (LUC) coding sequence under the control of the CBF3 promoter. Homozygous CBF3-LUC plants were chemically mutagenized and luminescence imaging isolated mutants with altered cold-induced CBF3-LUC expression. In the present specification, the Inventors report on the ice1 (for inducer of CBF expression 1) mutant, which is impaired in the cold-induction of CBF3-LUC and is defective in cold acclimation. ICE1 encodes a MYC-like basic helix-loop-helix transcriptional activator that binds to the CBF3 promoter. Thus, ICE1 plays a key role in regulating cold-responsive gene expression and cold tolerance in Arabidopsis.

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1. The ice1 mutation blocks the cold-induction of CBF3 and affects the expression of other cold-responsive genes. (A) Morphology (left) and CBF3-LUC luminescence images (right) of wild-type and ice1 seedlings. Luminescence images of the plants were collected after 12 h of cold (0° C.) treatment. (B) Quantitation of the luminescence intensities of wild type (solid circles) and ice1 (open circles) seedlings in response to different durations of cold treatment. (C) Transcript levels of CBFs and their downstream target genes in wild type and ice1 plants in response to cold treatment. Seedlings were either not treated (0 h) or treated with cold (0° C.) for the indicated durations (h). The tubulin gene was used as a loading control. WT, wild type.

FIG. 2. Morphology, and freezing and chilling sensitivity of ice1 mutant plants. (A) Wild type and ice1 seedlings in nutrient medium on agar under normal growth conditions. (B) Wild type and ice1 plants in soil under normal growth conditions. (C) ice1 plants are defective in cold acclimation. Ten-day-old seedlings grown at 22° C. were incubated for 4 days in light at 4° C. before freezing treatment at −12° C. The picture was taken 3 days after the freezing treatment. (D) Comparison of survival rates after freezing treatments at the indicated temperatures. Open circles and open triangles represent wild type and ice1 plants, respectively. (E) ice1 plants are sensitive to prolonged chilling treatment. After germination at 22° C., the plants were grown at 4° C. for 6 weeks. (F) Comparison of survival rates after 6 weeks of chilling stress.

FIG. 3. Confirmation of ICE1 gene cloning by expressing the dominant ice1 mutant allele in wild type plants. (A) Expression in wild type of a genomic fragment containing the ice1 mutation recapitulates the ice1 mutant phenotype. Seven-day-old seedlings of the wild type, ice1, and wild type transformed with the mutant ice1 gene grown on MS agar medium were subjected to luminescence imaging after 12 h of cold (0° C.) stress. (B) Quantitation of CBF3-LUC bioluminescence levels in wild type (WT), ice1 and WT transformed with the mutant ice1 gene after 12 h of cold (0° C.) stress.

FIG. 4. ICE1 encodes a bHLH protein. (A) Overall domain structure of ICE1 protein. A putative acidic domain (acidic), serine rich region (S-rich), bHLH domain, and possible zipper region (ZIP) are indicated. The arrow indicates the amino acid residue changed in the ice1 mutant. (B) Sequence alignment of the bHLH domains and ZIP regions of ICE1 and other plant and animal bHLH proteins. Identical and similar residues are shown in black and gray, respectively. A bold line indicates the basic region and open boxes connected with a loop indicate the helix-loop-helix domain. The zipper region is indicated as a dotted line. DDJB/EMBL/GenBank accession numbers and amino acid numbers (in parentheses) are: ICE1 (SEQ ID NO: 2), AY195621 (300-398); At1g12860 (SEQ ID NO: 3), NM_{—}101157 (638-731); At5g65640 (SEQ ID NO: 4), NM_{—}125962.1 (171-269); At5g10570 (SEQ ID NO: 5), NM_{—}121095.2 (144-242); rd22BP (SEQ ID NO: 6), AB000875 (446-544); ATR2 (SEQ ID NO: 7), NM_{—}124046.1 (409-507); maize R gene (SEQ ID NO: 8), M26227 (410-508); TT8 (SEQ ID NO: 9), AJ277509 (357-455); PIF3 (SEQ ID NO: 10), AF100166 (254-352); PIF4 (SEQ ID NO: 11), AJ440755 (255-353); MAX (SEQ ID NO: 12), P52161 (21-107); c-myc (SEQ ID NO: 13), 1001205A (354-435). Asterisks indicate amino acid residues of MAX that are known to interact with nucleotides (Grandori et al. 2000).

FIG. 5. Expression of the ICE1 gene and subcellular localization of the ICE1 protein. (A) ICE1 promoter driven GUS expression pattern in a wild type seedling. (B) ICE1 promoter-GUS expression in different plant tissues, and the corresponding ICE1 transcript levels as determined by RT-PCR analysis. The tubulin gene was used as an internal control in the RT-PCR. (C) RNA blot analysis of ICE1 expression in wild type seedlings under various abiotic stresses. Plants with the following treatments are shown: control, MS salt only; NaCl, 300 mM NaCl for 5 hr; ABA, 100 μM abscisic acid for 3 hr; Cold, 0° C. for 2 hr; Dehydration, air drying for 30 min. (D) Localization of GFP-ICE1 fusion protein in the nucleus. Panels (a)-(c) show confocal images of root cells in GFP-ICE1 transgenic plants, while panel (d) shows the location of nuclei as indicated by propidium stain.

FIG. 6. ICE1 protein binds to the MYC-recognition elements in the CBF3 promoter. (A) Sequences and positions of oligonucleotides within the CBF3 promoter used in the EMSA. Letters in bold indicate sequences of MYC-recognition motifs in MYC-1 (SEQ ID NO: 38), MYC-2 (SEQ ID NO: 37), MYC-3 (SEQ ID NO: 36), MYC-4 (SEQ ID NO: 35), and MYC-5 (SEQ ID NO: 34). Bold letters in the P1 (SEQ ID NO: 39) oligonucleotide are a putative MYB-recognition motif. The sequences labeled P2, MYC-2 (wt), and MYC-2 (M) correspond to (SEQ ID NO: 40), (SEQ ID NO: 41), and (SEQ ID NO: 42), respectively. (B) Interaction between ICE1 protein and ³²P-labeled MYC-1 through MYC-4 DNA fragments. (C) ICE1 binds to the MYC-2 DNA fragment more strongly than to the other DNA fragments. (D) Consensus nucleotide residues in the MYC-recognition motif are important for the interaction between ICE1 and the MYC-2 DNA fragment. (E) ice1 mutant protein also binds to the MYC-2 DNA fragment. The labeled oligonucleotides used in each experiment are indicated on the top of each panel. Triangles indicate increasing amounts of unlabeled oligonucleotides for competition in (B), (C) and (D), which correspond to 50-, 100- and 250-fold excess of each probe.

FIG. 7. ICE1 is a transcriptional activator and its overexpression enhances the CBF regulon in the cold and improves freezing tolerance. (A) Schematic representation of the reporter and effector plasmids used in the transient expression assay. A GAL4-responsive reporter gene was used in this experiment. Nos denotes the terminator signal of the nopaline synthase gene. Ω indicates the translational enhancer of tobacco mosaic virus. GAL4 DB is the DNA binding domain of the yeast transcription factor GAL4. (B) Relative luciferase activities after transfection with GAL4-LUC and 35S-GAL4-ICE1 or 35S-GAL4-ice1. To normalize values obtained after each transfection, a gene for luciferase from Renilla was used as an internal control. Luciferase activity is expressed in arbitrary units relative to the activity of Renilla luciferase (as described in Ohta et al. 2001). The values are averages of three bombardments, and error bars indicate standard deviations. (C) RNA blot analysis of ICE1 and cold responsive gene expression in wild type and ICE1 overexpressing transgenic (Super-ICE1 ) plants. Seedlings were either not treated (0 h) or treated with low temperature (0° C.) for 3 h or 6 h. Ethidium bromide stained rRNA bands are shown as loading control. (D) CBF3-LUC expression (indicated as luminescence intensity) in wild type and ICE1 overexpressing transgenic (Super-ICE1) plants. (E) Improved survival of ICE1 overexpressing transgenic (Super-ICE1) plants after a freezing treatment.

### DETAILED DESCRIPTION OF THE INVENTION

Unless specifically defined, all technical and scientific terms used herein have the same meaning as commonly understood by a skilled artisan in biochemistry, cellular biology, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory Press, New York (1982) and Sambrook et al., Molecular Cloning: A Laboratory Manual, Cold Spring Harbor Laboratory Press, New York (1989); Methods in Plant Molecular Biology, Maliga et al, Eds., Cold Spring Harbor Laboratory Press, New York (1995); Arabidopsis, Meyerowitz et al, Eds., Cold Spring Harbor Laboratory Press, New York (1994) and the various references cited therein.

The term “plant” includes whole plants, plant organs (e.g., leaves, stems, roots, etc.), seeds and plant cells and progeny of same. The class of plants, which can be used in the methods of the invention, is generally as broad as the class of higher plants amenable to transformation techniques, including both monocotyledonous and dicotyledonous plants. Preferred plants include rice, corn, wheat, cotton, peanut, and soybean.

Thus, in one embodiment of the present invention, cold acclimation can be enhanced or increased by increasing the amount of protein available in the plant, preferably by the enhancement of the ice1 gene in the plant.

Thus, one embodiment of the present invention is plant cells carrying the polynucleotides of the present invention, and preferably transgenic plants carrying the isolated polynucleotides of the present invention.

As used herein, the term “enhancement” means increasing the intracellular activity of one or more enzymes in a plant cell and/or plant, which are encoded by the corresponding DNA. Enhancement can be achieved with the aid of various manipulations of the bacterial cell. In order to achieve enhancement, particularly over-expression, the number of copies of the corresponding gene can be increased, a strong promoter can be used, or the promoter- and regulation region or the ribosome binding site which is situated upstream of the structural gene can be mutated. Expression cassettes which are incorporated upstream of the structural gene may act in the same manner. In addition, it is possible to increase expression by employing inducible promoters. A gene can also be used which encodes a corresponding enzyme with a high activity. Expression can also be improved by measures for extending the life of the mRNA. Furthermore, preventing the degradation of the enzyme increases enzyme activity as a whole. Moreover, these measures can optionally be combined in any desired manner. These and other methods for altering gene activity in a plant are known as described, for example, in Methods in Plant Molecular Biology, Maliga et al, Eds., Cold Spring Harbor Laboratory Press, New York (1995).

An “expression cassette” as used herein includes a promoter, which is functional in a plant cell, operably linked to an isolated nucleic acid encoding an ICE1 protein of SEQ ID NO: 2, wherein enhanced expression of the protein in a plant cell imparts increased cold acclimation to said plant cell. In a preferred embodiment of the present invention the promoter is selected from the group consisting of a viral coat protein promoter, a tissue-specific promoter, a monocot promoter, a ubiquitin promoter, a stress inducible promoter, a CaMV 35S promoter, a CaMV 19S promoter, an actin promoter, a cab promoter, a sucrose synthase promoter, a tubulin promoter, a napin R gene complex promoter, a tomato E8 promoter, a patatin promoter, a mannopine synthase promoter, a soybean seed protein glycinin promoter, a soybean vegetative storage protein promoter, a bacteriophage SP6 promoter, a bacteriophage T3 promoter, a bacteriophage T7 promoter, a Ptac promoter, a root-cell promoter, an ABA-inducible promoter and a turgor-inducible promoter.

A gene can also be used which encodes a corresponding or variant enzyme with a high activity. Preferably the corresponding enzyme has a greater activity than the native form of the enzyme, more preferably at least in the range of 5, 10, 25% or 50% more activity, most preferably more than twice the activity of the native enzyme.

In the context of the present Application, a polynucleotide sequence is “homologous” with the sequence according to the invention if at least 70%, preferably at least 80%, most preferably at least 90% of its base composition and base sequence corresponds to the sequence according to the invention. According to the invention, a “homologous protein” is to be understood to comprise proteins which contain an amino acid sequence at least 70% of which, preferably at least 80% of which, most preferably at least 90% of which, corresponds to the amino acid sequence which is shown in SEQ ID NO: 2 or which is encoded by the ice1 gene (SEQ ID No.1), wherein corresponds is to be understood to mean that the corresponding amino acids are either identical or are mutually homologous amino acids. The expression “homologous amino acids” denotes those that have corresponding properties, particularly with regard to their charge, hydrophobic character, steric properties, etc. Thus, the protein may be from 70% up to less than 100% homologous to SEQ ID NO: 2.

Homology, sequence similarity or sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

The present invention also relates to polynucleotides which contain the complete gene with the polynucleotide sequence corresponding to SEQ ID NO: 1 or fragments thereof, and which can be obtained by screening by means of the hybridization of a corresponding gene bank with a probe which contains the sequence of said polynucleotide corresponding to SEQ ID NO: 1 or a fragment thereof, and isolation of said DNA sequence.

Polynucleotide sequences according to the invention are suitable as hybridization probes for RNA, cDNA and DNA, in order to isolate those cDNAs or genes which exhibit a high degree of similarity to the sequence of the ice1 gene, in particular the ice1 gene of SEQ ID NO: 1.

Polynucleotide sequences according to the invention are also suitable as primers for polymerase chain reaction (PCR) for the production of DNA which encodes an enzyme having ICE1 transcriptional activator activity.

Oligonucleotides such as these, which serve as probes or primers, can contain more than 30, preferably up to 30, more preferably up to 20, most preferably at least 15 successive nucleotides. Oligonucleotides with a length of at least 40 or 50 nucleotides are also suitable.

The term “isolated” means separated from its natural environment.

The term “polynucleotide” refers in general to polyribonucleotides and polydeoxyribonucleotides, and can denote an unmodified RNA or DNA or a modified RNA or DNA.

The term “polypeptides” is to be understood to mean peptides or proteins, which contain two or more amino acids which are bound via peptide bonds.

The polypeptides according to invention include polypeptides corresponding to SEQ ID NO: 2, particularly those with the biological activity of a ICE1 transcriptional activator, and also includes those, at least 70% of which, preferably at least 80% of which, are homologous with the polypeptide corresponding to SEQ ID NO: 2, and most preferably those which exhibit a homology of least 90% to 95% with the polypeptide corresponding to SEQ ID NO: 2 and which have the cited activity. Thus, the polypeptides may have a homology of from 70% up to 100% with respect to SEQ ID NO: 2.

The invention also relates to coding DNA sequences, which result from SEQ ID NO: 1 by degeneration of the genetic code. In the same manner, the invention further relates to DNA sequences which hybridize with SEQ ID NO: 1 or with parts of SEQ ID NO: 1. Moreover, one skilled in the art is also aware of conservative amino acid replacements such as the replacement of glycine by alanine or of aspartic acid by glutamic acid in proteins as “sense mutations” which do not result in any fundamental change in the activity of the protein, i.e. which are functionally neutral. It is also known that changes at the N- and/or C-terminus of a protein do not substantially impair the function thereof, and may even stabilize said function.

In the same manner, the present invention also relates to DNA sequences which hybridize with SEQ ID NO: 1 or with parts of SEQ ID NO: 1. Finally, the present invention relates to DNA sequences which are produced by polymerase chain reaction (PCR) using oligonucleotide primers which result from SEQ ID NO: 1. Oligonucleotides of this type typically have a length of at least 15 nucleotides.

The terms “stringent conditions” or “stringent hybridization conditions” includes reference to conditions under which a polynucleotide will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tm can be approximated from the equation of Meinkoth and Wahl, Anal. Biochem., 138:267-284 (1984): Tm=81.5oC.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with approximately 90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution) it is preferred to increase the SSC concentration so that a higher temperature can be used. An extensive guide to the hybridization of nucleic acids is found in Current Protocols in Molecular Biology, Chapter 2, Ausubel, et al., Eds., Greene Publishing and Wiley-Interscience, New York (2000).

Thus, with the foregoing information, the skilled artisan can identify and isolated polynucleotides that are substantially similar to the present polynucleotides. In so isolating such a polynucleotide, the polynucleotide can be used as the present polynucleotide in, for example, increasing cold acclimation of a plant.

One embodiment of the present invention is methods of screening for polynucleotides that have substantial homology to the polynucleotides of the present invention, preferably those polynucleotides encoding a protein having ICE1 transcriptional activator activity.

The polynucleotide sequences of the present invention can be carried on one or more suitable plasmid vectors, as known in the art for plants or the like.

In one embodiment, it may be advantageous for propagating the polynucleotide to carry it in a bacterial or fungal strain with the appropriate vector suitable for the cell type. Common methods of propagating polynucleotides and producing proteins in these cell types are known in the art and are described, for example, in Maniatis et al., Molecular Cloning: A Laboratory Manual, Cold Spring Harbor Laboratory Press, New York (1982) and Sambrook et al., Molecular Cloning: A Laboratory Manual, Cold Spring Harbor Laboratory Press, New York (1989).

In another preferred embodiment the polynucleotide comprises SEQ ID NO: 1, polynucleotides which are complimentary to SEQ ID NO: 1, polynucleotides which are at least 70%, 80% and 90% identical to SEQ ID NO: 1; or those sequence which hybridize under stringent conditions to SEQ ID NO: 1, the stringent conditions comprise washing in 5×SSC at a temperature from 50 to 68° C. Thus, the polynucleotide may be from 70% up to less than 100% identical to SEQ ID NO: 1.

In another preferred embodiment the polynucleotides of the present invention are in a vector and/or a host cell. Preferably, the polynucleotides are in a plant cell or transgenic plant. Preferably, the plant is Arabidopsis thaliania or selected from the group consisting of wheat, corn, peanut cotton, oat, and soybean plant. In a preferred embodiment, the polynucleotides are operably linked to a promoter, preferably an inducible promoter.

In another preferred embodiment the present invention provides, a process for screening for polynucleotides which encode a protein having ICE1 transcriptional activator activity comprising hybridizing the polynucleotide of the invention to the polynucleotide to be screened; expressing the polynucleotide to produce a protein; and detecting the presence or absence of ICE1 transcriptional activator activity in the protein.

In another preferred embodiment, the present invention provides a method for detecting a nucleic acid with at least 70% homology to nucleotide SEQ ID NO: 1, sequences which are complimentary to SEQ ID NO: 1 and/or which encode a protein having the amino acid sequence in SEQ ID NO: 2 comprising contacting a nucleic acid sample with a probe or primer comprising at least 15 consecutive nucleotides of the nucleotide sequence of SEQ ID NO: 1, or at least 15 consecutive nucleotides of the complement thereof.

In another preferred embodiment, the present invention provides a method for producing a nucleic acid with at least 70% homology to the polynucleotides of the present invention comprising contacting a nucleic acid sample with a primer comprising at least 15 consecutive nucleotides of the nucleotide sequence of SEQ ID NO: 1, or at least 15 consecutive nucleotides of the complement thereof.

In another preferred embodiment, the present invention provides a method for making ICE1 protein, comprising culturing the host cell carrying the polynucleotides of the invention for a time and under conditions suitable for expression of ICE1, and collecting the ICE1.

In another preferred embodiment, the present invention provides a method of making a transgenic plant comprising introducing the polynucleotides of the invention into the plant.

In another preferred embodiment, the present invention provides method of increasing cold acclimation of a plant in need thereof, comprising introducing the polynucleotides of the invention into said plant.

Methods, vectors, and compositions for transforming plants and plant cells in accordance with the invention are well-known to those skilled in the art, and are not particularly limited. For a descriptive example see Karimi et al., TRENDS in Plant Science, Vol. 7, NO: 5, May 2002, pp. 193-195, incorporated herein by reference.

In another preferred embodiment, the present invention provides an isolated polypeptide comprising the amino acid sequence in SEQ ID NO: 2 or those proteins that are at least 70%, preferably 80%, preferably 90% and preferably 95% identity to SEQ ID NO: 2, where the polypeptides have ICE1 transcriptional activator activity. Thus, the enzyme has a homology of from 70% to less than 100% homology to SEQ ID NO: 2.

In another embodiment, the present invention also provides a method of increasing cold acclimation in a plant, comprising overexpressing an ICE1 transcriptional activator in the plant.

The present invention also provides, in another embodiment a method of increasing cold acclimation in a plant by increasing the expression of one or more additional transcription factors selected from the group consisting of a CBF transcription factor and a DREB1 transcription factor and/or by increasing expression of one or more cold-responsive genes.

In the context of the present invention the term “cold responsive genes” include genes that encode a protein selected from the group consisting of an enzyme involved in respiration of carbohydrates, an enzyme involved in metabolism of carbohydrates, an enzyme involved in respiration of lipids, an enzyme involved in metabolism of lipids, an enzyme involved in respiration of phenylpropanoids, an enzyme involved in metabolism of phenylpropanoids, an enzyme involved in respiration of antioxidants, an enzyme involved in metabolism of antioxidants, a molecular chaperone, an antifreeze protein, and a protein involved in tolerance to the dehydration caused by freezing.

The present invention has been accomplished using a genetic screen (Chinnusamy et al. 2002) to identify cold signaling components upstream of the CBF proteins. A cold-responsive bioluminescent Arabidpsis plant was engineered by expressing the firefly luciferase (LUC) coding sequence under the control of the CBF3 promoter. Homozygous CBF3-LUC plants were chemically mutagenized and luminescence imaging isolated mutants with altered cold-induced CBF3-L UC expression. In the present specification, the Inventors report on the ice1 (for inducer of CBF expression 1) mutant, which is impaired in the cold-induction of CBF3-LUC and is defective in cold acclimation. ICE1 encodes a MYC-like basic helix-loop-helix transcriptional activator that binds to the CBF3 promoter. Thus, ICE1 plays a key role in regulating cold-responsive gene expression and cold tolerance in Arabidopsis.

Discussion

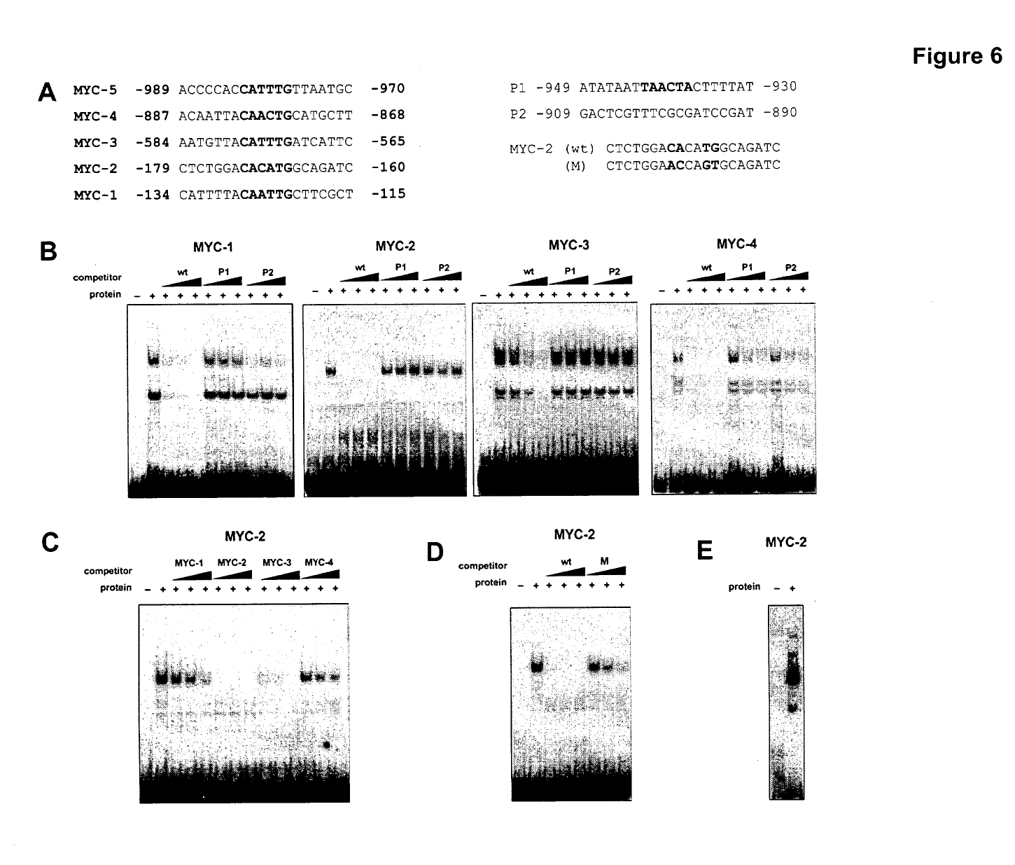

Cold temperatures trigger the transcription of the CBF family of transcription factors, which in turn activate the transcription of genes containing the DRE/CRT promoter element (Thomashow 1999). The CBF target genes presumably include some transcription factors (Fowler and Thomashow 2002). Therefore, cold signaling for freezing tolerance requires a cascade of transcriptional regulations. In the present study, we have identified ICE1, a very upstream transcription factor of this cascade. Our results show that ICE1 is a positive regulator of CBF3 and has a critical role in cold acclimation. ICE1 encodes a MYC-like bHLH transcription factor. Five putative MYC recognition sequences are present in the CBF3 promoter, while CBF1 and CBF2 promoters each contain one such element (Shinwari et al. 1998). This is consistent with the fact that CBF3 is more strongly affected by the ice1 mutation than are CBF1 or CBF2. DNA binding assays showed that ICE1 can specifically bind to the MYC recognition sequences on the CBF3 promoter but not to a putative MYB recognition sequence (FIG. 6). The ice1 mutation abolishes CBF3 expression, and reduces the expression of CBF-target genes in the cold. Consistent with its role in cold-responsive gene regulation, ICE1 is important for chilling and freezing tolerance of Arabidopsis plants.

The ice1 mutation also affects the cold-induction of CBF1 and CBF2; their expression is slightly reduced early in the cold, but at later time points the expression is not reduced. Instead, the expression of CBF2 is actually enhanced in the ice1 mutant after 6 and 12 hours of cold treatment. The expression of CBF genes is known to be repressed by their gene products or the products of their downstream target genes (Guo et al. 2002). The correlation between the reduced CBF3 expression and enhanced CBF2 induction suggests that CBF3 may repress CBF2 expression. When the CBF2 gene is disrupted, CBF1 and CBF3 show more sustained induction in the cold (Julio Salinas, personal communication), suggesting that CBF2 may repress the expression of CBF1 and CBF3. The potential negative regulation of each other among the CBF transcription factor genes may be important for ensuring that their expression is transient and tightly controlled.

The three CBF genes are generally presumed to be functionally redundant. Their individual contribution has not been examined by loss of function analysis. Even though the ice1 mutation only blocks the expression of CBF3, the downstream genes such as RD29A, COR15A and COR47 are substantially affected. This suggests that CBF3 plays a critical role in the cold regulation of these genes. In comparison, the cold regulation of KIN1 is less affected by the ice1 mutation. Therefore, it is possible that the three CBF genes may each have their own set of preferred target genes.

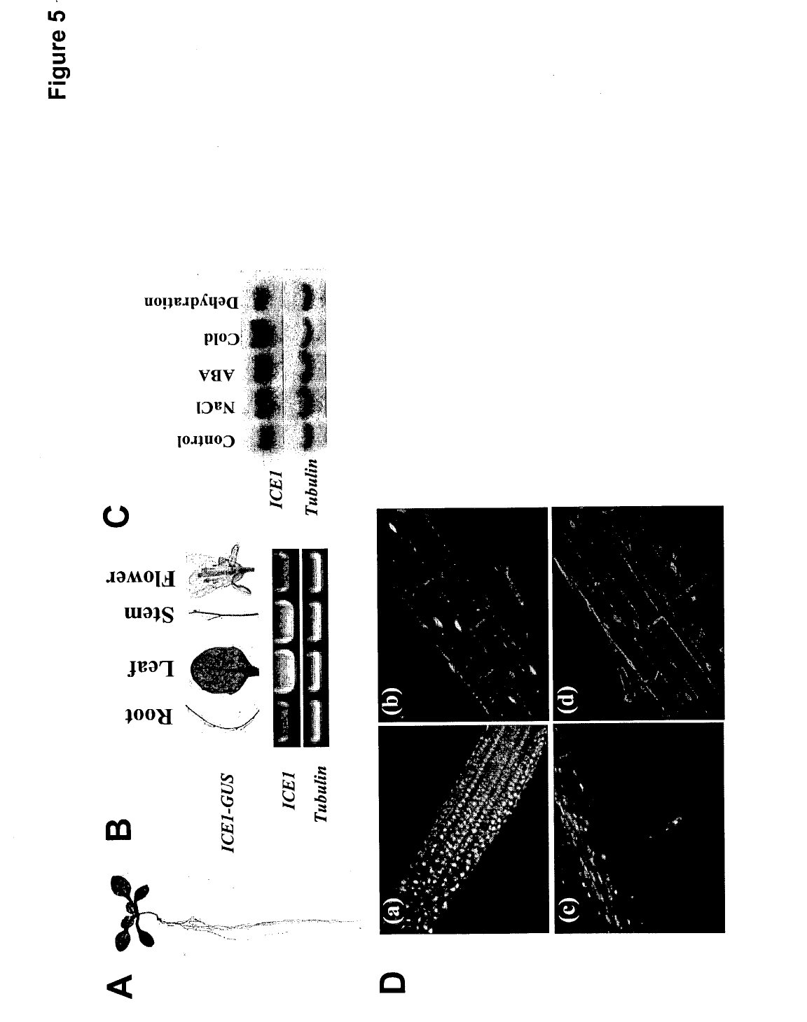

ICE1 is expressed constitutively in all tissues (FIGS. 5A and 5B), and is only slightly up-regulated by cold (FIG. 5C). Consistent with what has been speculated for “ICE” proteins. (Gilmour et al. 1998), cold induced modification of the ICE1 protein or of a transcriptional co-factor appears to be necessary for ICE1 to activate the expression of CBFs. Our evidence supports this because ICE1 is expressed constitutively and localized in the nucleus, but the CBF expression requires cold treatment; and transgenic lines constitutively overexpressing ICE1 do not show CBF3 expression at warm temperatures but have a higher level of CBF3 expression at cold temperatures. The ability of transcription factors to activate gene transcription may be regulated by protein phosphorylation and dephosphorylation in the cytoplasm or in the nucleus (reviewed by Liu et al. 1999). The ice1 mutation is very near potential serine phosphorylation residues (Ser243 and Ser245), and thus might affect the phosphorylation/dephosphorylation of ICE1.

It is known that MYC-related bHLH transcription factors require MYB co-transcription factors and/or WD-repeat containing factors for transcriptional activation of target genes (Spelt et al. 2000; Walker et al. 1999). The promoters of CBFs contain MYC as well as potential MYB recognition sequences (Shinwari et al. 1998), suggesting that a MYB-related transcription factor may also be involved in the cold induction of CBFs. The ice1 mutation, which substitutes Arg236 with His, may interfere with hetero-oligomer formation between ICE1 and an ICE1-like protein or a MYB-related co-factor. Alternatively, the putative dominant negative effect of ice1 could be a consequence of ice1 interference with potential ICE1 homo-oligomer formation, protein stability, nuclear localization, or cold induced post-translational modification of ICE1.

Having generally described this invention, a further understanding can be obtained by reference to certain specific examples, which are provided herein for purposes of illustration only, and are not intended to be limiting unless otherwise specified.

### EXAMPLES

Materials and Methods

Plant Materials and Mutant Isolation:

The CBF3 promoter, a region from 1126 to 100 bp upstream of the initiation codon, was obtained by polymerase chain reaction (PCR) using the following primer pair: 5′-TCATGGATCCACCATTTGTTAATGCATGATGG-3′(SEQ ID NO: 14) and 5′-GCTCAAGCTTTCTGTTCTAGTTCAGG-3′(SEQ ID NO: 15). This promoter was placed in front of the firefly luciferase (LUC) coding sequence in a plant transformation vector (Ishitani et al. 1997). Arabidopsis thaliana ecotype Columbia (with the glabrous1 mutation) was transformed with Agrobacterium tumefaciens containing this CBF3-LUC construct by the floral dipping method. Plants homozygous for the CBF3-LUC transgene were selected from the second generation after transformation. One such plant with a single copy of the CBF3-LUC transgene was chosen for subsequent experiments (hereafter referred to as wild type). This wild type plant did not show any bioluminescence when grown under normal growth conditions, but emitted bioluminescence when cold stress was imposed. The CBF3-LUC plant seeds were mutagenized with ethyl methanesulfonate (EMS). Seedlings of the M2 generation were used to screen for mutants defective in cold regulated CBF3-LUC expression by luminescence imaging. Seven-day-old seedlings grown on 0.6% agar plates containing 3% sucrose and 1×Murashige and Skoog (MS) salts (JRH Biosciences) were screened for de-regulated luciferase expression in response to low temperature treatment at 0° C. for 12 hours, using a low light video imaging system (Princeton Instruments). Luminescence intensities of individual seedlings were quantified with the WINVIEW software provided by the camera manufacturer (Princeton Instruments) (Chinnusamy et al. 2002).

Chilling and Freezing Tolerance Assays:

Chilling sensitivity of ice1 and wild type plants were tested by exposing the seedlings immediately after radicle emergence. After 2 days of stratification at 4° C., mutant and wild-type seeds were germinated at 22° C. on MS nutrient medium with 3% sucrose and 1.2% agar. Chilling stress was imposed by incubating the seedlings at 4±1° C. with 30±2 μmol quanta. m⁻².s⁻¹ light. Freezing tolerance was assayed as described (Xin and Browse, 1998). Briefly, wild type and ice1 seeds were sown on agar (0.9%) plates with Gamborg basal salts and 1.5% sucrose. After 2 days of stratification at 4° C., the plates were kept at 22° C. under 50±2 μmol quanta m⁻².s⁻¹ continuous light. Ten-day-old seedlings were cold acclimated at 4±1° C. and 30±2 μmol quanta. m⁻².s⁻¹ light for 4 days. These plants on petri dishes were placed on ice in a freezing chamber (Percival Scientific) set to −1±0.1° C. for 16 h. Ice chips were sprinkled on these plants before the chamber was programmed to cool at 1° C. h⁻¹. Petri dishes of plants were removed after being frozen at desired temperatures for 2 hours unless indicated otherwise, thawed at 4° C. for 12 hours in the dark, and then transferred to 22° C. under 50±2 μmol quanta m⁻².s⁻¹ continuous light. Survival of the seedlings was scored visually after two days.

Gene Expression Analysis:

For RNA analysis, ten-day-old seedlings of WT and ice1 plants grown on separate halves of the same MS agar plates were used. Total RNA extracted from control and stressed plants was analyzed by RNA blotting as described by Liu and Zhu (1997). The RD29A gene-specific probe was from the 3′noncoding region (Liu and Zhu 1997). COR15A and COR47 cDNAs (Gilmour et al. 1992; Lin and Thomashow 1992) were kindly provided by M. F. Thomashow (Michigan State University). The CBF2 and CBF3 gene-specific probes were generated by PCR with the following primer pairs: CBF2-forward primer, 5′-TTCGATTTTTATTTCCATTTTTGG-3′(SEQ ID NO: 16); CBF2-reverse primer, 5′-CCAAACGTCCTTGAGTCTTGAT-3′(SEQ ID NO: 17); CBF3-forward primer, 5′-TAAAACTCAGATTATTATTTCCATTT-3′(SEQ ID NO: 18); CBF3-reverse primer, 5′-GAGGAGCCACGTAGAGGGCC-3′(SEQ ID NO: 19). The probe for KINI (Kurkela and Franck, 1990) was a 0.4-kb EcoR1 fragment of the Arabidopsis EST clone YAP368T7. The β-tubulin gene was used as a loading control and was amplified by PCR with the following primer pairs: forward primer (5′-CGTGGATCACAGCAATACAGAGCC-3′(SEQ ID NO: 20)) and reverse primer (5′-CCTCCTGCACTTCCACTTCGTCTTC-3′(SEQ ID NO: 21)).

For Affymetrix GeneChip array analysis, 20 μg of total RNA from the wild type and ice1 seedlings with or without cold treatment (6 hours under light) were extracted using the RNeasy Plant Mini Kit (Qiagen) and used to make biotin-labeled CRNA targets. The Affymetrix Arabidopsis ATHI genome array GeneChips, which contain more than 22,500 probe sets representing approximately 24,000 genes, were used and hybridization, washing, and staining were carried out as directed in the manufacturer's manual. Microarray data were extracted from scanned GeneChip images and analyzed using Microarray Suite version 5.0.1 (Affymetrix).

Mapping and Cloning of the ICE1 Locus:

Genetic analysis of F₁ and F₂ progenies of the ice1 cross with WT showed that ice1 is a dominant mutation. Hence to clone ICE1, a homozygous ice1 plant was crossed with the Arabidopsis Landsberg erecta (Ler) ecotype and the F₂ progeny from self-pollinated F₁ were used to select mapping samples with the wild type phenotype. Genomic DNA extracted from these seedlings was used for PCR-based mapping with simple sequence polymorphism markers or cleaved amplified polymorphic sequence markers. New SSLP mapping markers on F16J4, MTC11, MLJ15, MDJ14, K17E12 and T32N 15BAC clones were developed based on insertion/deletions identified from the Cereon Arabidopsis polymorphism and Ler sequence collection (http://www.arabidopsis.org). Genomic DNA corresponding to candidate genes was amplified by PCR from ice1 mutant and wild type plants and sequenced to identify the ice1 mutation.

For ice1 mutant complementation, the MLJ15.14 gene, including 2,583 bp upstream of the initiation codon and 615 bp downstream of the stop codon, was PCR amplified by LA Taq polymerase (Takara) using ice1 mutant genomic DNA as template. The PCR primers used were as follows: forward primer: 5′-AGGGATCCGGACCACCGTCAATAACATCGTTAAGTAG-3′(SEQ ID NO: 22); reverse primer: 5′-CGAATTCTAACCGCCATTAACTATGTCTCCTCTCTATCTC-3′(SEQ ID NO: 23). The resulting 5,035-bp fragment was T-A cloned into the pCR2.1 TOPO vector (Invitrogen) and then subcloned into pCAMBIA1200 between the BamHI and EcoRI sites. This and all other constructs described here were completely sequenced to ensure that they did not contain PCR or cloning errors. The binary construct was then introduced into Agrobacterium strain GV3101 and transformed into CBF3-LUC Columbia wild type plants. Hygromycin-resistant transgenic plants were selected and their T2 progenies were tested for CBF3-LUC expression in response to cold stress.

Analysis of ICE1 Expression:

The promoter region (2,589 bp upstream from the initiation codon) of the ICE1 gene was PCR-amplified with the following primer pair: forward primer, 5′-AGGGATCCGGACCACCGTCAATAACATCGTTAAGTAG-3′(SEQ ID NO: 24); reverse primer, 5′-CGAATTCGCCAAAGTTGACACCTTTACCCCAAAG-3′(SEQ ID NO: 25). The resulting fragment was digested with BamHI and EcoRI and inserted into the pCAMBIA1391 binary vector. This ICE1 promoter-GUS construct was introduced into Agrobacterium strain GV3101 and transformed into wild type Arabidopsis. T2 transgenic lines resistant to hygromycin were analyzed for ICE1-promoter driven GUS expression. For GUS staining, T2 seedlings grown on MS agar plates were incubated with X-Gluc. for 12 h at 37° C. and then washed 5 times with 70% (v/v) ethanol at 70° C. to remove chlorophyll. ICE1 expression was also examined by quantitative RT-PCR analysis of RNA prepared from wild type roots, leaves, stems and flowers. The ICE1 cDNA was amplified by RT-PCR using the following primers: forward primer: 5′-GCGATGGGTCTTGACGGAAACAATGGTG-3′ (SEQ ID NO: 26) and reverse primer 5′-TCAGATCATACCAGCATACCCTGCTGTATCG-3′(SEQ ID NO: 27). The tubulin gene was used as an internal control in the RT-PCR analysis. Tubulin cDNA was amplified using the following primers: forward primer: 5′-GTCAAGAGGTTCTCAGCAGTA-3′ (SEQ ID NO: 28) and reverse primer 5′-TCACCTTCTTGATCCGCAGTT-3′ (SEQ ID NO: 29).

Overexpression of ICE1:

The ICE1 cDNA was amplified from Arabidopsis (ecotype Columbia) RNA by RT-PCR using the following primers: a forward primer: 5′-GCTCTAGAGCGATGGGTCTTGACGGAAACAATGGTG-3′(SEQ ID NO: 30) and a reverse primer 5′-GGGGTACCTCAGATCATACCAGCATACCCTGCTGTATCG-3′(SEQ ID NO: 31). The PCR product was digested with XbaI and KpnI, and cloned into the pBIB vector under control of the superpromoter, which consists of three copies of the octopine synthase upstream-activating sequence in front of the manopine synthase promoter (Li et al. 2001). Agrobacterium tumefaciens strain GV3101 containing this binary construct was used to transform Arabidopsis plants. Transformants were selected on MS medium containing hygromycin (30 mg/l).

Expression and Localization of GFP-ICE1 Fusion Protein:

The full-length ICE1 cDNA was obtained from wild type plants by RT-PCR using the following primers: forward primer, 5′-AGGAATTCGCGATGGGTCTTGACGGAAACAATGGTG-3′(SEQ ID NO: 32); reverse primer, 5′-CTGGATCCTCAGATCATACCAGCATACCCTGCTGTATCG-3′(SEQ ID NO: 33). The resulting PCR fragment was digested with EcoRI and BamHI and cloned into the binary vector pEGAD downstream from the CaMV 35S promoter. This GFP-ICE1 construct was introduced into Agrobacterium strain GV3101 and transformed into wild type Arabidopsis. T2 transgenic lines resistant to Basta (glufosinate) were selected and analyzed for GFP expression. To visualize the nucleus, root tissues were stained with propidium iodide (1 μg/mL). Green fluorescence (GFP expression) and red fluorescence (propidium iodide staining) analyses of transgenic plants were performed with a confocal laser-scanning microscope.

DNA Binding Assay:

The wild type and mutant ICE1 cDNAs were amplified by RT-PCR and inserted into NdeI and BamHI sites in the expression vector pET14b (Novagen). Wild-type and mutant His-ICE1 fusion proteins were prepared from E. coli cells (BL21 DE3) according to the instruction manual of His-Bind Buffer Kit (Novagen). The electrophoresis mobility shift assay (EMSA) was carried out as described (Hao et al. 1998). The following double-stranded oligonucleotides listed in FIG. 6A (MYC-1, MYC-2, MYC-3, MYC-4 and MYC-5) were used as probes and competitors in EMSAs. Nucleotide sequences P1 (−949 to −930) and P2 (−909 to −890) were also used as competitors. P1 contains a putative MYB-recognition site. P2 does not contain any typical cis-elements. DNA probes were end-labeled with [γ-³²P]dCTP using the Klenow fragment and purified through a Sephadex G-50 column. The labeled probes (ca 0.02 pmol) were incubated for 20 min at room temperature with 2.3 μg of purified His-ICE1 fusion protein in 1×binding buffer (Hao et a.1998) supplemented with 20 pmol poly(dI-dC). The resulting DNA-protein complexes were resolved by electrophoresis on a 6% polyacrylamide gel in 0.5×TBE buffer and visualized by autoradiography. For competition experiments, unlabeled competitors were incubated with the His-ICE1 fusion protein on ice for 30 min prior to the addition of labeled probes.

Transient Expression Assay:

The wild type (ICE1) and mutant (ice1) cDNAs were amplified by RT-PCR, digested with SalI and inserted into SmaI and SalI sites of the plant expression vector 35S-GAL4 DB (Ohta et al. 2000). The plasmid DNA of the resulting effector, GAL4-ICE1, and a GAL4 responsive reporter, GAL4-LUC (Ohta et al. 2000) were delivered into Arabidopsis leaves using particle bombardment (Ohta et al. 2001).

### Experimental Example

Identification of the ICE1 Locus:

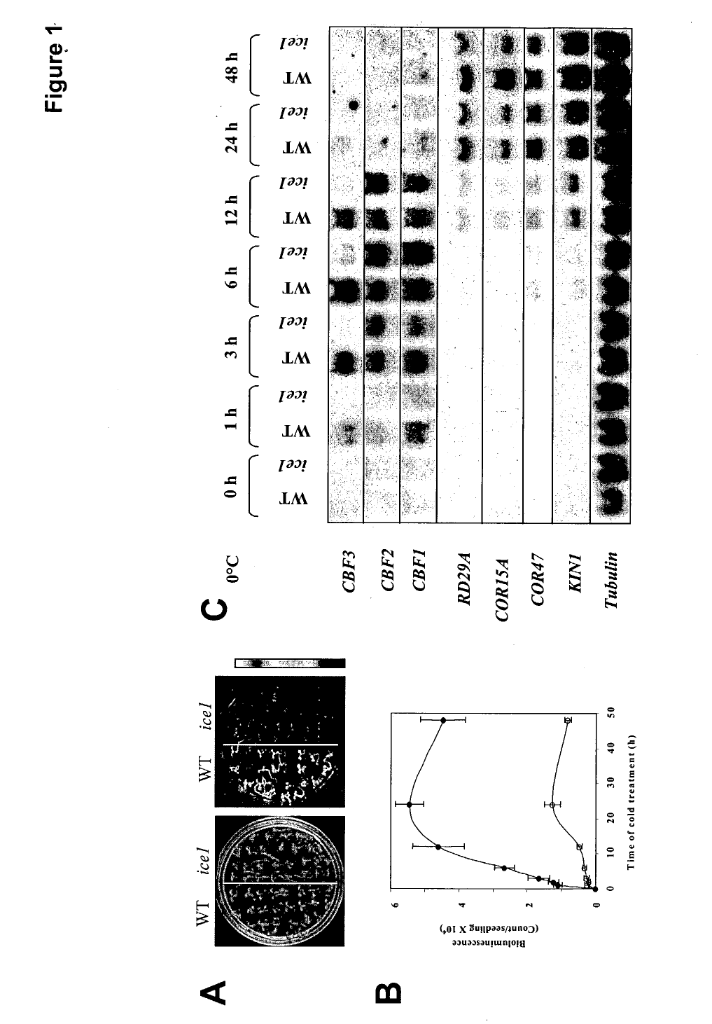

Using the genetic screen noted above, Arabidopsis plants containing the CBF3-LUC transgene emitted bioluminescence in response to cold stress (FIGS. 1A and 1B). The homozygous CBF3-LUC plants (herein referred to as wild type) were mutagenized by ethylmethane sulfonate, and the resulting M2 population was screened for mutants with aberrant bioluminescence responses under cold stress using a low light imaging system (Chinnusamy et al. 2002). Several mutants showing abnormal cold regulation of CBF3-LUC expression were recovered. One of these mutant lines, designated as ice1, is virtually blocked in CBF3-LUC expression in the cold (FIGS. 1A and 1B). In response to treatment at 0° C., wild type plants showed strong luminescence, while the ice1 mutant showed very little induction of luminescence throughout the duration of cold treatment (FIGS. 1A and 1B). After 12 hours of cold treatment, ice1 plants showed nearly 10 times less luminescence than that of wild type plants, and are obviously defective in the cold regulation of CBF3-L UC expression (FIG. 1B).

The ice1 mutant plant was crossed with CBF3-LUC wild type plants and the resulting F1 plants were examined for CBF3-LUC expression after 12 hours of cold treatment at 0° C. As determined by luminescence imaging, all F1 plants showed reduced cold-induced CBF3-LUC expression similar to that of ice1. An F2 population from the selfed F1 segregated in an approximately 3 to 1 ratio between mutant and wild type. These results show that ice1 is a dominant mutation in a single nuclear gene.

ice1 Mutant Plants are Defective in Cold-Regulated Gene Expression:

RNA blot analysis was carried out to analyze the effect of ice1 mutation on the transcript levels of endogenous CBFs and their target cold stress-responsive genes. Consistent with the imaging results, cold induction of the endogenous CBF3 gene was greatly impaired (almost abolished) in ice1 mutant plants (FIG. 1C). Wild type plants showed CBF3 induction after 1 hour of cold stress and the expression peaked at 6 hour. In contrast, CBF3 induction was almost abolished in ice1 plants (FIG. 1C). While the CBF1 induction level was lower in the ice1 mutant was lower than that of wild type at 1 and 3 hours of cold stress, its induction level at 6 and 12 hours was similar to that in the wild type. The CBF2 induction level was slightly lower in ice1 at 1 hour of cold treatment, whereas at 6 and 12 hours, the induction level was higher in the mutant (FIG. 1C). We also examined the cold induction of the downstream target genes of CBFs. The expression levels of RD29A, COR15A and COR47A under cold stress were lower in ice1 than in the wild type, while the induction of KIN1 was lower in ice1 only after 48 hours of cold stress (FIG. 1C).

Consistent with these RNA blot results, microarray analysis using Affymetrix near full genome genechips showed that out of 306 genes induced 3-fold or more in the wild type by a 6-hour cold treatment, 217 are either not induced in the ice1 mutant or their induction is 50% or less of that in the wild type (Table 1A). Thirty-two of these encode putative transcription factors, suggesting that ICE1 may control many cold-responsive regulons. For 87 of the 306 cold induced genes, their induction levels in the wild type and ice1 differ by less than 2-fold (Table 1B). Interestingly, 2 genes show higher levels of cold induction in the ice1 mutant (Table 1C).

Table 1. Cold-Responsive Gene Expression in the Wild Type and ice1.

For cold treatment, the wild-type and ice1 seedlings were placed at 0±1° C. under light for 6 hours. Affymetrix GeneChip analysis was carried out as described in materials and methods. Gene expression changes were analyzed by comparing values for a cold-treated sample to those for a control sample in each genotype. ‘Fold Change’ value of +1 or −1 indicates no change in gene expression. Up-regulation or down-regulation is expressed by either + or − in ‘Fold Change’ values, respectively. Cold-responsive genes were determined in the wild type by the following standards; 1) signal intensities from cold treated sample were greater than background (i.e. genes with ‘Present’ calls, determined by Affymetrix Microarray Suite Program, in a cold treated sample); 2) ‘Change’ calls, made by Affymetrix Microarray Suite, in pair-comparison were ‘I’(for ‘increase’); 3) the ‘Fold Change’ in pair-comparison was 3-fold or higher. The expression of the resulting 306 genes was further analyzed and compared with that in ice1 mutant. A two-fold difference between changes in the wild type and ice1 was used as a threshold to categorize genes. Transcription factors are shown in gray blocks. Genes used for RNA hybridization analysis are in bold. The fold change values for 22 genes in cold-treated ice1 were not determined (ND) because their signal intensities were similar to the background value (i.e. genes with ‘Absent’ calls in cold-treated ice1). These 22 genes were all cold-induced in the wild type. Therefore, they were included in the category of cold-responsive genes with lower induction in ice1 than in the wild type.

TABLE 1A
Cold-responsive genes with lower induction in ice1

| | | | Fold Change | |
|---|---|---|---|---|
| Probe Set | AGI ID | Gene Title | WT | ice1 |
| 254074_at | At4g25490 | CBF1/DREB1B | 445.7 | 64.0 |
| 254066_at | At4g25480 | CBF3/DREB1A | 78.8 | 29.9 |
| 258325_at | At3g22830 | putative heat shock transcription factor1 | 42.2 | 5.7 |
| 246432_at | At5g17490 | RGA-like protein | 34.3 | ND |
| 261648_at | At1g27730 | salt-tolerance zinc finger protein | 24.3 | 6.5 |
| 247655_at | At5g59820 | zinc finger protein Zat12 | 19.7 | 7.0 |
| 248160_at | At5g54470 | CONSTANS B-box zinc finger family protein | 19.7 | 9.8 |
| 250781_at | At5g05410 | DRE binding protein (DREB2A) | 14.9 | 4.9 |
| 251745_at | At3g55980 | Zn finger transcription factor (PE11) | 13.9 | 3.0 |
| 258139_at | At3g24520 | heat shock transcription factor HSF1, putative | 13.9 | 3.7 |
| 245711_at | At5g04340 | putative c2h2 zinc finger transcription factor | 11.3 | 5.3 |
| 245250_at | At4g17490 | ethylene-responsive element binding factor 6 (AtERF6) | 8.6 | 4.3 |
| 252214_at | At3g50260 | EREBP-3 homolog | 8.6 | 2.1 |
| 261613_at | At1g49720 | abscisic acid responsive elements-binding factor | 7.0 | 3.5 |
| 245078_at | At2g23340 | putative AP2 domain transcription factor | 5.7 | 1.4 |
| 263379_at | At2g40140 | putative CCCH-type zinc finger protein | 5.7 | 2.6 |
| 253405_at | At4g32800 | transcription factor TINY, putative | 5.3 | ND |
| 245807_at | At1g46768 | AP2 domain protein RAP2.1 | 4.9 | 1.9 |
| 259432_at | At1g01520 | myb family transcription factor | 4.9 | 2.3 |
| 252278_at | At3g49530 | NAC2-like protein | 4.6 | 2.0 |
| 253485_at | At4g31800 | WRKY family transcription factor | 4.6 | −1.2 |
| 251272_at | At3g61890 | homeobox-leucine zipper protein ATHB-12 | 4.3 | 1.3 |
| 261470_at | At1g28370 | ethylene-responsive element binding factor 11 (AtERF11) | 4.3 | 1.7 |
| 261892_at | At1g80840 | WRKY family transcription factor | 4.3 | 1.2 |
| 263783_at | At2g46400 | WRKY family transcription factor | 4.3 | 1.4 |
| 257022_at | At3g19580 | zinc finger protein, putative | 3.7 | 1.4 |
| 267252_at | At2g23100 | CHP-rich zinc finger protein, putative | 3.7 | ND |
| 249746_at | At5g24590 | NAC2-like protein | 3.5 | 1.6 |
| 256093_at | At1g20823 | putative RING zinc finger protein | 3.5 | 1.4 |
| 252009_at | At3g52800 | zinc finger-like protein | 3.2 | 1.3 |
| 256185_at | At1g51700 | Dof zinc finger protein | 3.2 | 1.6 |
| 260763_at | At1g49220 | RING-H2 finger protein RHA3a, putative | 3.2 | ND |
| 245749_at | At1g51090 | proline-rich protein, putative | 73.5 | 7.5 |
| 264217_at | At1g60190 | hypothetical protein | 68.6 | 26.0 |
| 246467_at | At5g17040 | UDP glucose:flavonoid 3-o-glucosyltransferase-like protein | 29.9 | ND |
| 251793_at | At3g55580 | regulator of chromosome condensation-like protein | 27.9 | 6.1 |
| 262452_at | At1g11210 | expressed protein | 27.9 | 7.0 |
| 264661_at | At1g09950 | hypothetical protein | 27.9 | ND |
| 258947_at | At3g01830 | expressed protein | 26.0 | 2.5 |
| 246178_s_at | At5g28430 | putative protein | 19.7 | 7.0 |
| 253104_at | At4g36010 | thaumatin-like protein | 19.7 | 2.5 |
| 257391_at | At2g32050 | hypothetical protein | 19.7 | ND |
| 245627_at | At1g56600 | water stress-induced protein, putative | 18.4 | ND |
| 247208_at | At5g64870 | nodulin-like | 18.4 | 1.2 |
| 256114_at | At1g16850 | expressed protein | 18.4 | 2.6 |
| 256356_s_at | At1g66500 | hypothetical protein | 18.4 | 3.0 |
| 250098_at | At5g17350 | putative protein | 17.1 | 3.2 |
| 264758_at | At1g61340 | late embryogenesis abundant protein, putative | 17.1 | 5.3 |
| 246099_at | At5g20230 | blue copper binding protein | 16.0 | 1.3 |
| 257280_at | At3g14440 | 9-cis-epoxycarotenoid dioxygenase (neoxanthin cleavage | 16.0 | 1.0 |
| | | enzyme) (NC1) (NCED1), putative | | |
| 251336_at | At3g61190 | putative protein | 13.9 | 3.0 |
| 260264_at | At1g68500 | hypothetical protein | 13.9 | 3.0 |
| 263497_at | At2g42540 | COR15a | 13.9 | 3.5 |
| 248337_at | At5g52310 | RD29A/COR78/LTI78 | 13.0 | 4.6 |
| 248959_at | At5g45630 | putative protein | 13.0 | 2.0 |
| 259977_at | At1g76590 | expressed protein | 13.0 | 2.5 |
| 260399_at | At1g72520 | putative lipoxygenase | 13.0 | 1.5 |
| 259879_at | At1g76650 | putative calmodulin | 12.1 | 2.8 |
| 265290_at | At2g22590 | putative anthocyanidin-3-glucoside rhamnosyltransferase | 12.1 | ND |
| 267411_at | At2g34930 | disease resistance protein family | 12.1 | 1.1 |
| 250648_at | At5g06760 | late embryogenesis abundant protein LEA like | 11.3 | 1.9 |
| 257876_at | At3g17130 | hypothetical protein | 11.3 | 2.3 |
| 260727_at | At1g48100 | polygalacturonase, putative | 11.3 | 2.3 |
| 246125_at | At5g19875 | Expressed protein | 10.6 | 2.0 |
| 251603_at | At3g57760 | putative protein | 10.6 | 1.1 |
| 256017_at | At1g19180 | expressed protein | 10.6 | 1.5 |
| 264617_at | At2g17660 | unknown protein | 10.6 | ND |
| 264787_at | At2g17840 | putative senescence-associated protein 12 | 10.6 | 3.5 |
| 245757_at | At1g35140 | phosphate-induced (phi-1) protein, putative | 9.8 | 1.6 |
| 252346_at | At3g48650 | hypothetical protein | 9.8 | 2.0 |
| 253643_at | At4g29780 | expressed protein | 9.8 | 3.2 |
| 254667_at | At4g18280 | glycine-rich cell wall protein-like | 9.8 | 1.2 |
| 264389_at | At1g11960 | unknown protein | 9.8 | 1.7 |
| 266545_at | At2g35290 | hypothetical protein | 9.8 | 1.4 |
| 266720_s_at | At2g46790 | expressed protein | 9.8 | 4.9 |
| 245251_at | At4g17615 | calcineurin B-like protein 1 | 9.2 | 3.0 |
| 247431_at | At5g62520 | putative protein | 9.2 | 1.5 |
| 248964_at | At5g45340 | cytochrome p450 family | 9.2 | 3.0 |
| 252368_at | At3g48520 | cytochrome p450, putative | 9.2 | 1.3 |
| 262164_at | At1g78070 | expressed protein | 9.2 | 4.3 |
| 252102_at | At3g50970 | dehydrin Xero2 | 8.6 | 4.0 |
| 262359_at | At1g73070 | leucine rich repeat protein family | 8.6 | ND |
| 262731_at | At1g16420 | hypothetical protein common family | 8.6 | 2.8 |
| 245677_at | At1g56660 | hypothetical protein | 8.0 | 2.8 |
| 245734_at | At1g73480 | lysophospholipase homolog, putative | 8.0 | 2.6 |
| 247177_at | At5g65300 | expressed protein | 8.0 | 3.7 |
| 250053_at | At5g17850 | potassium-dependent sodium-calcium exchanger-like protein | 8.0 | 2.0 |
| 254120_at | At4g24570 | mitochondrial carrier protein family | 8.0 | 3.0 |
| 254926_at | At4g11280 | ACC synthase (AtACS-6) | 8.0 | 2.3 |
| 263789_at | At2g24560 | putative GDSL-motif lipase/hydrolase | 8.0 | ND |
| 245346_at | At4g17090 | glycosyl hydrolase family 14 (beta-amylase) | 7.5 | 2.6 |
| 253425_at | At4g32190 | putative protein | 7.5 | 3.2 |
| 254085_at | At4g24960 | abscisic acid-induced-like protein | 7.5 | 2.3 |
| 259076_at | At3g02140 | expressed protein | 7.5 | 1.1 |
| 260227_at | At1g74450 | expressed protein | 7.5 | 2.0 |
| 260915_at | At1g02660 | expressed protein | 7.5 | 1.7 |
| 262677_at | At1g75860 | unknown protein | 7.5 | 2.6 |
| 266532_at | At2g16890 | putative glucosyltransferase | 7.5 | 3.0 |
| 247925_at | At5g57560 | xyloglucan endotransglycosylase (TCH4) | 7.0 | 2.1 |
| 252563_at | At3g45970 | putative protein | 7.0 | 1.2 |
| 254850_at | At4g12000 | putative protein | 7.0 | 2.3 |
| 260744_at | At1g15010 | expressed protein | 7.0 | 1.7 |
| 263931_at | At2g36220 | expressed protein | 7.0 | 3.0 |
| 245306_at | At4g14690 | Expressed protein | 6.5 | 2.6 |
| 246495_at | At5g16200 | putative protein | 6.5 | 1.7 |
| 248870_at | At5g46710 | putative protein | 6.5 | 2.6 |
| 253292_at | At4g33985 | Expressed protein | 6.5 | 2.0 |
| 253872_at | At4g27410 | putative protein | 6.5 | 1.5 |
| 258792_at | At3g04640 | expressed protein | 6.5 | 3.0 |
| 259516_at | At1g20450 | expressed protein | 6.5 | 3.2 |
| 262050_at | At1g80130 | expressed protein | 6.5 | 1.2 |
| 245427_at | At4g17550 | putative protein | 6.1 | 1.2 |
| 253859_at | At4g27657 | Expressed protein | 6.1 | ND |
| 261187_at | At1g32860 | glycosyl hydrolase family 17 | 6.1 | 1.4 |
| 262448_at | At1g49450 | En/Spm-like transposon protein, putative | 6.1 | ND |
| 266757_at | At2g46940 | unknown protein | 6.1 | 1.3 |
| 252131_at | At3g50930 | BCS1 protein-like protein | 5.7 | 1.6 |
| 255795_at | At2g33380 | RD20 protein | 5.7 | −1.3 |
| 258321_at | At3g22840 | early light-induced protein | 5.7 | 2.3 |
| 262496_at | At1g21790 | expressed protein | 5.7 | 2.0 |
| 265119_at | At1g62570 | flavin-containing monooxygenase, putative | 5.7 | 2.0 |
| 246018_at | At5g10695 | Expressed protein | 5.3 | 1.7 |
| 248820_at | At5g47060 | putative protein | 5.3 | 1.7 |
| 249918_at | At5g19240 | putative protein | 5.3 | 1.9 |
| 253830_at | At4g27652 | Expressed protein | 5.3 | 1.7 |
| 246490_at | At5g15950 | S-adenosylmethionine decarboxylase (adoMetDC2) | 4.9 | 2.1 |
| 253284_at | At4g34150 | putative protein | 4.9 | 1.9 |
| 253323_at | At4g33920 | putative protein | 4.9 | 2.5 |
| 253614_at | At4g30350 | putative protein | 4.9 | 1.5 |
| 264655_at | At1g09070 | expressed protein | 4.9 | 1.9 |
| 245533_at | At4g15130 | putative phosphocholine cytidylyltransferase | 4.6 | 2.1 |
| 246831_at | At5g26340 | hexose transporter-like protein | 4.6 | 2.0 |
| 247137_at | At5g66210 | calcium-dependent protein kinase | 4.6 | 1.4 |
| 247226_at | At5g65100 | putative protein | 4.6 | ND |
| 250467_at | At5g10100 | trehalose-6-phosphate phosphatase-like protein | 4.6 | ND |
| 252414_at | At3g47420 | putative protein | 4.6 | 1.9 |
| 252997_at | At4g38400 | putative pollen allergen | 4.6 | 1.1 |
| 253595_at | At4g30830 | putative protein | 4.6 | ND |
| 253832_at | At4g27654 | Expressed protein | 4.6 | 1.3 |
| 258188_at | At3g17800 | expressed protein | 4.6 | 1.4 |
| 259479_at | At1g19020 | Expressed protein | 4.6 | 1.7 |
| 261405_at | At1g18740 | expressed protein | 4.6 | 2.0 |
| 262881_at | At1g64890 | expressed protein | 4.6 | 2.1 |
| 264000_at | At2g22500 | mitochondrial carrier protein family | 4.6 | 2.0 |
| 265668_at | At2g32020 | putative alanine acetyl transferase | 4.6 | 2.3 |
| 265797_at | At2g35715 | Expressed protein | 4.6 | ND |
| 248686_at | At5g48540 | 33 kDa secretory protein-like | 4.3 | 1.6 |
| 250676_at | At5g06320 | harpin-induced protein-like | 4.3 | 1.6 |
| 251259_at | At3g62260 | protein phosphatase 2C (PP2C) | 4.3 | 2.1 |
| 254300_at | At4g22780 | Translation factor EF-1 alpha-like protein | 4.3 | −1.1 |
| 261356_at | At1g79660 | unknown protein | 4.3 | 1.6 |
| 264636_at | At1g65490 | expressed protein | 4.3 | 1.4 |
| 246468_at | At5g17050 | UDP glucose:flavonoid 3-o-glucosyltransferase-like protein | 4.0 | 2.0 |
| 248607_at | At5g49480 | NaCl-inducible Ca2+-binding protein-like; calmodulin-like | 4.0 | 1.5 |
| 250279_at | At5g13200 | ABA-responsive protein-like | 4.0 | 1.2 |
| 252053_at | At3g52400 | syntaxin SYP122 | 4.0 | 1.9 |
| 256633_at | At3g28340 | unknown protein | 4.0 | 2.0 |
| 258207_at | At3g14050 | putative GTP pyrophosphokinase | 4.0 | 1.7 |
| 258805_at | At3g04010 | glycosyl hydrolase family 17 | 4.0 | 1.3 |
| 261912_s_at | At1g66000 | hypothetical protein | 4.0 | ND |
| 264989_at | At1g27200 | expressed protein | 4.0 | 1.6 |
| 265276_at | At2g28400 | hypothetical protein | 4.0 | −1.1 |
| 267261_at | At2g23120 | expressed protein | 4.0 | 1.7 |
| 247693_at | At5g59730 | putative protein | 3.7 | 1.9 |
| 253113_at | At4g35985 | putative protein | 3.7 | 1.4 |
| 253165_at | At4g35320 | putative protein | 3.7 | 1.9 |
| 253879_s_at | At4g27570 | UDP rhamnose-anthocyanidin-3-glucoside rhamnosyltransferase-like protein | 3.7 | 1.2 |
| 253915_at | At4g27280 | putative protein | 3.7 | 1.9 |
| 259426_at | At1g01470 | hypothetical protein | 3.7 | 1.6 |
| 259445_at | At1g02400 | dioxygenase, putative | 3.7 | 1.9 |
| 260410_at | At1g69870 | putative peptide transporter | 3.7 | 1.1 |
| 261581_at | At1g01140 | serine threonine kinase, putative | 3.7 | 1.5 |
| 262113_at | At1g02820 | late embryogenis abundant protein, putative | 3.7 | 1.1 |
| 262382_at | At1g72920 | disease resistance protein (TIR-NBS class), putative | 3.7 | 1.9 |
| 265665_at | At2g27420 | cysteine proteinase | 3.7 | 1.0 |
| 267069_at | At2g41010 | unknown protein | 3.7 | 1.2 |
| 245450_at | At4g16880 | disease resistance RPP5 like protein (fragment) | 3.5 | ND |
| 246289_at | At3g56880 | putative protein | 3.5 | 1.3 |
| 249204_at | At5g42570 | expressed protein | 3.5 | 1.5 |
| 249622_at | At5g37550 | putative protein | 3.5 | 1.4 |
| 250335_at | At5g11650 | lysophospholipase-like protein | 3.5 | 1.7 |
| 251372_at | At3g60520 | putative protein | 3.5 | 1.5 |
| 254707_at | At4g18010 | putative protein | 3.5 | 1.1 |
| 257154_at | At3g27210 | expressed protein | 3.5 | 1.5 |
| 259705_at | At1g77450 | GRAB1-like protein | 3.5 | 1.3 |
| 261037_at | At1g17420 | lipoxygenase | 3.5 | 1.2 |
| 261937_at | At1g22570 | peptide transporter, putative | 3.5 | 1.6 |
| 264024_at | At2g21180 | expressed protein | 3.5 | 1.1 |
| 264458_at | At1g10410 | unknown protein | 3.5 | 1.2 |
| 266799_at | At2g22860 | unknown protein | 3.5 | 1.4 |
| 247280_at | At5g64260 | phi-1-like protein | 3.2 | 1.6 |
| 251356_at | At3g61060 | putative protein | 3.2 | 1.5 |
| 252316_at | At3g48700 | putative protein | 3.2 | 1.3 |
| 253824_at | At4g27940 | putative protein | 3.2 | 1.1 |
| 256526_at | At1g66090 | disease resistance protein (TIR-NBS class), putative | 3.2 | 1.4 |
| 256595_x_at | At3g28530 | hypothetical protein | 3.2 | 1.1 |
| 265648_at | At2g27500 | glycosyl hydrolase family 17 | 3.2 | 1.1 |
| 266097_at | At2g37970 | expressed protein | 3.2 | 1.6 |
| 267335_s_at | At2g19440 | glycosyl hydrolase family 17 | 3.2 | 1.6 |
| 245699_at | At5g04250 | putative protein | 3.0 | 1.2 |
| 247467_at | At5g62130 | putative protein | 3.0 | 1.5 |
| 249583_at | At5g37770 | CALMODULIN-RELATED PROTEIN 2, TOUCH-INDUCED (TCH2) | 3.0 | 1.1 |
| 249626_at | At5g37540 | putative protein | 3.0 | 1.2 |
| 252474_at | At3g46620 | putative protein | 3.0 | 1.3 |
| 253628_at | At4g30280 | xyloglucan endotransglycosylase, putative | 3.0 | 1.4 |
| 253835_at | At4g27820 | glycosyl hydrolase family 1 | 3.0 | 1.2 |
| 254158_at | At4g24380 | putative protein | 3.0 | 1.4 |
| 254188_at | At4g23920 | UDPglucose 4-epimerase like protein | 3.0 | 1.2 |
| 254634_at | At4g18650 | putative protein | 3.0 | ND |
| 254973_at | At4g10460 | putative retrotransposon | 3.0 | ND |
| 256763_at | At3g16860 | unknown protein | 3.0 | 1.0 |
| 257519_at | At3g01210 | RRM-containing protein | 3.0 | −1.1 |
| 258894_at | At3g05650 | disease resistance protein family | 3.0 | 1.4 |
| 265841_at | At2g35710 | putative glycogenin | 3.0 | 1.5 |
| 266271_at | At2g29440 | glutathione transferase, putative | 3.0 | 1.2 |
| 266316_at | At2g27080 | expressed protein | 3.0 | 1.1 |
| 267631_at | At2g42150 | hypothetical protein | 3.0 | 1.1 |

TABLE 1B
Cold-responsive genes with similar induction in the wild type and ice1

| | | | Fold Change | |
|---|---|---|---|---|
| Probe Set | AGI ID | Gene Title | WT | ice1 |
| 254075_at | At4g25470 | CBF2/DREB1C | 104.0 | 137.2 |
| 261263_at | At1g26790 | Dof zinc finger protein | 68.6 | 55.7 |
| 257262_at | At3g21890 | CONSTANS B-box zinc finger family protein | 7.5 | 5.3 |
| 259834_at | At1g69570 | Dof zinc finger protein | 7.0 | 5.7 |
| 256430_at | At3g11020 | DREB2B | 6.1 | 4.9 |
| 248389_at | At5g51990 | DRE binding protein | 5.3 | 4.0 |
| 257053_at | At3g15210 | AtERF4 | 5.3 | 2.8 |
| 248744_at | At5g48250 | CONSTANS B-box zinc finger family protein | 4.9 | 3.0 |
| 249606_at | At5g37260 | CCA1, putative | 4.9 | 9.2 |
| 267028_at | At2g38470 | WRKY family transcription factor | 4.9 | 3.0 |
| 246523_at | At5g15850 | CONSTANS-LIKE 1 | 4.0 | 3.2 |
| 248799_at | At5g47230 | AtERF5 | 4.0 | 3.5 |
| 247452_at | At5g62430 | Dof zinc finger protein | 3.7 | 3.7 |
| 251190_at | At3g62690 | RING-H2 zinc finger protein ATL5 | 3.7 | 2.1 |
| 253722_at | At4g29190 | Zn finger protein, putative | 3.7 | 4.6 |
| 259992_at | At1g67970 | putative heat shock transcription factor | 3.7 | 2.3 |
| 263252_at | At2g31380 | CONSTANS-like B-box zinc finger protein | 3.7 | 3.7 |
| 263739_at | At2g21320 | CONSTANS B-box zinc finger family protein | 3.7 | 2.3 |
| 252429_at | At3g47500 | Dof zinc finger protein | 3.5 | 4.3 |
| 253140_at | At4g35480 | RING-H2 finger protein RHA3b | 3.5 | 2.0 |
| 258742_at | At3g05800 | bHLH protein | 3.5 | 6.5 |
| 265939_at | At2g19650 | CHP-rich zinc finger protein, putative | 3.5 | 2.8 |
| 249415_at | At5g39660 | Dof zinc finger protein | 3.2 | 3.7 |
| 259364_at | At1g13260 | DNA-binding protein (RAV1) | 3.2 | 2.1 |
| 262590_at | At1g15100 | putative RING-H2 zinc finger protein | 3.2 | 2.0 |
| 263823_s_at | At2g40350 | AP2 domain transcription factor | 3.0 | 5.7 |
| 264511_at | At1g09350 | putative galactinol synthase | 17.1 | 12.1 |
| 264314_at | At1g70420 | expressed protein | 13.0 | 9.8 |
| 247478_at | At5g62360 | DC1.2 homologue-like protein | 11.3 | 11.3 |
| 253322_at | At4g33980 | putative protein | 11.3 | 8.0 |
| 249741_at | At5g24470 | putative protein | 8.0 | 6.5 |
| 247047_at | At5g66650 | putative protein | 7.0 | 4.3 |
| 263495_at | At2g42530 | COR15b | 6.5 | 9.8 |
| 265536_at | At2g15880 | unknown protein | 6.5 | 5.3 |
| 249174_at | At5g42900 | putative protein | 6.1 | 3.5 |
| 249191_at | At5g42760 | putative protein | 6.1 | 4.3 |
| 264153_at | At1g65390 | disease resistance protein (TIR class), putative | 6.1 | 4.9 |
| 250099_at | At5g17300 | expressed protein | 5.7 | 7.5 |
| 265725_at | At2g32030 | putative alanine acetyl transferase | 5.7 | 3.0 |
| 246922_at | At5g25110 | serine/threonine protein kinase-like protein | 4.9 | 4.9 |
| 251494_at | At3g59350 | protein kinase-like protein | 4.9 | 2.8 |
| 246821_at | At5g26920 | calmodulin-binding protein | 4.6 | 2.6 |
| 255733_at | At1g25400 | expressed protein | 4.6 | 3.0 |
| 257650_at | At3g16800 | protein phosphatase 20 (PP2C) | 4.6 | 2.5 |
| 266832_at | At2g30040 | putative protein kinase | 4.6 | 2.8 |
| 267357_at | At2g40000 | putative nematode-resistance protein | 4.6 | 3.7 |
| 249411_at | At5g40390 | glycosyl hydrolase family 36 | 4.3 | 3.5 |
| 256266_at | At3g12320 | expressed protein | 4.3 | 4.0 |
| 252956_at | At4g38580 | copper chaperone (CCH)-related | 4.0 | 2.3 |
| 253455_at | At4g32020 | putative protein | 4.0 | 2.6 |
| 259570_at | At1g20440 | hypothetical protein | 4.0 | 2.3 |
| 262383_at | At1g72940 | disease resistance protein (TIR-NBS class), putative | 4.0 | 2.1 |
| 247393_at | At5g63130 | unknown protein | 3.7 | 3.2 |
| 252661_at | At3g44450 | putative protein | 3.7 | 2.5 |
| 259990_s_at | At1g68050 | F-box protein FKF1/ADO3, AtFBX2a | 3.7 | 2.3 |
| 264213_at | At1g65400 | hypothetical protein | 3.7 | 2.0 |
| 245777_at | At1g73540 | unknown protein | 3.5 | 2.5 |
| 248745_at | At5g48260 | unknown protein | 3.5 | 2.5 |
| 248846_at | At5g46500 | putative protein | 3.5 | 2.6 |
| 249063_at | At5g44110 | ABC transporter family protein | 3.5 | 2.1 |
| 257654_at | At3g13310 | DnaJ protein, putative | 3.5 | 2.1 |
| 257925_at | At3g23170 | expressed protein | 3.5 | 1.9 |
| 261048_at | At1g01420 | flavonol 3-o-glucosyltransferase, putative | 3.5 | 2.0 |
| 263216_s_at | At1g30720 | FAD-linked oxidoreductase family | 3.5 | 2.3 |
| 265184_at | At1g23710 | expressed protein | 3.5 | 2.3 |
| 245558_at | At4g15430 | hypothetical protein | 3.2 | 3.5 |
| 248164_at | At5g54490 | putative protein | 3.2 | 2.5 |
| 248502_at | At5g50450 | putative protein | 3.2 | 4.3 |
| 252010_at | At3g52740 | expressed protein | 3.2 | 2.5 |
| 253679_at | At4g29610 | cytidine deaminase 6 (CDA6) | 3.2 | 2.0 |
| 256548_at | At3g14770 | expressed protein | 3.2 | 1.9 |
| 256577_at | At3g28220 | unknown protein | 3.2 | 2.3 |
| 257083_s_at | At3g20590 | non-race specific disease resistance protein, putative | 3.2 | 2.3 |
| 260046_at | At1g73800 | Expressed protein | 3.2 | 2.0 |
| 261958_at | At1g64500 | peptide transporter, putative | 3.2 | 2.6 |
| 263352_at | At2g22080 | En/Spm-like transposon protein | 3.2 | 1.9 |
| 263452_at | At2g22190 | putative trehalose-6-phosphate phosphatase | 3.2 | 2.3 |
| 265093_at | At1g03905 | ABC transporter family protein | 3.2 | 1.7 |
| 267293_at | At2g23810 | hypothetical protein | 3.2 | 1.7 |
| 245119_at | At2g41640 | expressed protein | 3.0 | 2.6 |
| 246270_at | At4g36500 | putative protein | 3.0 | 3.0 |
| 247793_at | At5g58650 | putative protein | 3.0 | 1.7 |
| 256442_at | At3g10930 | expressed protein | 3.0 | 1.9 |
| 256487_at | At1g31540 | disease resistance protein (TIR-NBS-LRR class), putative | 3.0 | 2.1 |
| 259428_at | At1g01560 | MAP kinase, putative | 3.0 | 1.7 |
| 266834_s_at | At2g30020 | protein phosphatase 2C (PP2C) | 3.0 | 2.6 |
| 267364_at | At2g40080 | expressed protein | 3.0 | 2.3 |

TABLE 1C
Cold responsive genes with higher induction in ice1

| | | | Fold Change | |
|---|---|---|---|---|
| Probe Set | AGI ID | Gene Title | WT | ice1 |
| 261248_at | At1g20030 | calreticulin, putative | 4.6 | 13.9 |
| 258383_at | At3g15440 | hypothetical protein | 4.3 | 9.2 |

The ice1 Mutation Impairs Chilling and Freezing Tolerance

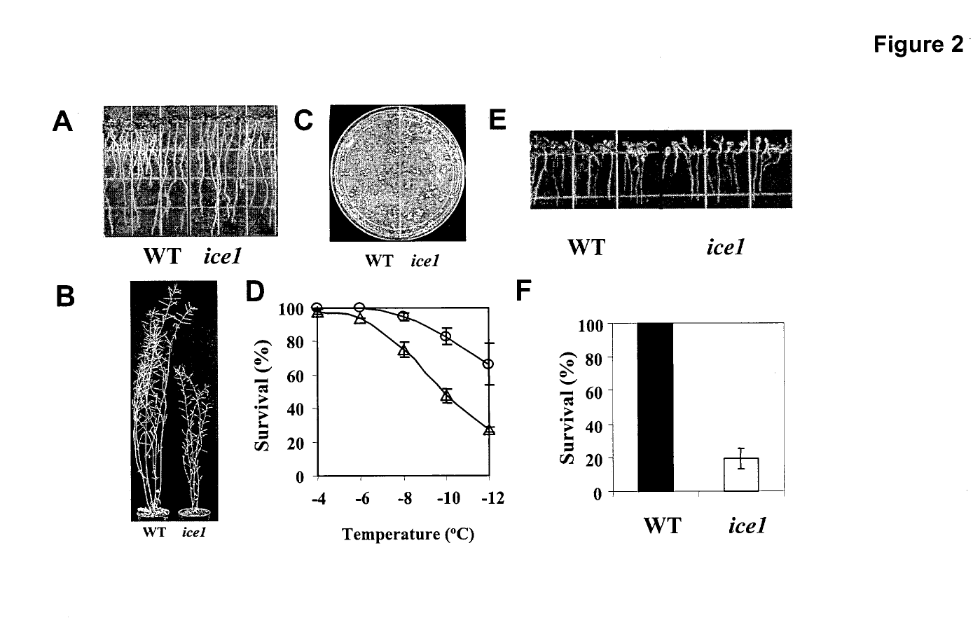

At normal growth temperatures, ice1 and wild type seedlings were similar in size (FIG. 2A). Although adult ice1 plants were smaller, they were not very different from the wild type in flowering time and fertility (FIG. 2B). Ten-day-old seedlings of ice1 and wild type grown on separate halves of the same agar plates were cold acclimated at 4° C. for four days and then subjected to a freezing tolerance assay. The ice1 mutant was less freezing-tolerant than the wild type at all freezing temperatures (FIGS. 2C and 2D). Freezing at −10° C. for 2 hours killed about 50% of ice1 mutant plants whereas less than 20% of wild type plants were killed at this temperature (FIG. 2D). When newly germinated (at 22° C.) ice1 and wild type seedlings were transferred to 4° C. (with 30±2 μmol quanta. m⁻².s⁻¹ light), chilling injury became apparent in the mutant after 4 weeks of cold treatment (FIG. 2E). After 6 weeks of chilling stress, 100% of wild type but only 20% of ice1 mutant plants survived (FIG. 2F).

Positional Cloning of ICE1

To map the ice1 mutation, a homozygous ice1 mutant in the CBF3-LUC Columbia background was crossed to wild type plants of the Ler ecotype. F1 plants from the cross were selfed to produce F2 seeds. Since the ice1 mutation is dominant, we selected from the segregating F2 population seedlings with the wild type phenotype (based on plant size and morphology) for mapping. A total of 662 wild type plants were selected and used for mapping with simple sequence length polymorphism and cleaved amplified polymorphic sequence markers (see Materials and Methods section for details), which initially placed ice1 on the middle of chromosome 3, then narrowed its location to a 58 kb region on the MLJ15 and MDJ14 BAC clones. Candidate genes in this region were amplified from homozygous ice1 mutant plants and sequenced. The sequences were compared with the published sequence of Arabidopsis ecotype Columbia and a single G to A mutation in the hypothetical MLJ15.14 gene was found.

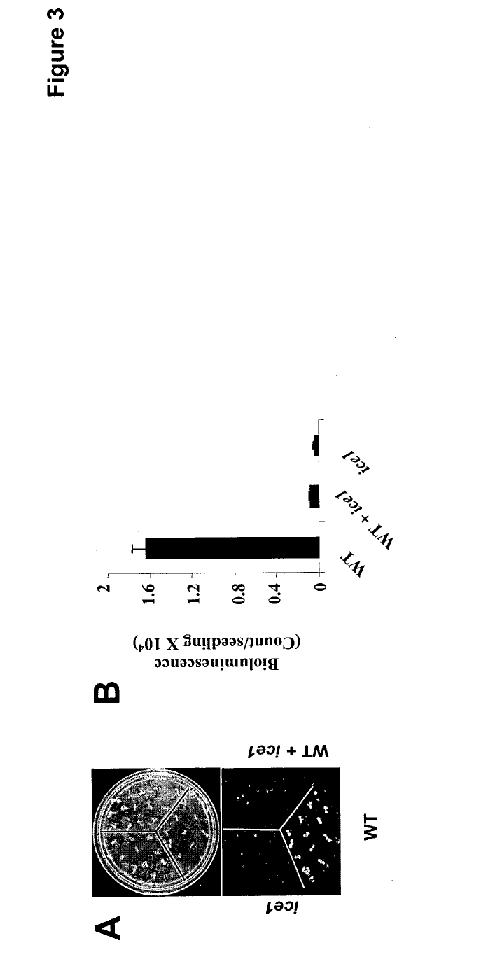

To confirm that MLJ15.14 is the ICE1 gene, the MLJ15.14 gene including 2,583 bp upstream of the initiation codon and 615 bp downstream of the stop codon was cloned from ice1 mutant plants. This fragment was inserted into a binary vector and introduced into CBF3-LUC Columbia wild type plants by Agrobacterium-mediated transformation. Transgenic plants were selected based on their hygromycin resistance, and cold-induced bioluminescence in the T2 lines was compared with that of the wild type. The MLJ 15.14 gene from ice1 suppressed cold-induced luminescence from the wild type plants (FIGS. 3A and 3B) and reduced the plant height to that of ice1 mutant, thus confirming that MLJ 15.14 is ICE1.

ICE1 Encodes a Constitutively Expressed and Nuclear Localized MYC-Like Basic Helix-Loop-Helix Transcription Factor

The open reading frame of ICE1 (SEQ ID NO: 1)was determined by sequencing cDNAs obtained by RT-PCR. The open reading frame was determined to be:

| | |
|---|---|
| 1 | atcaaaaaaa aagtttcaat ttttgaaagc tctgagaaat gaatctatca ttctctctct |
| 61 | ctatctctat cttccttttc agatttcgct tcttcaattc atgaaatcct cgtgattcta |
| 121 | ctttaatgct tctctttttt tacttttcca agtctctgaa tattcaaagt atatatcttt |
| 181 | tgttttcaaa cttttgcaga attgtcttca agcttccaaa tttcagttaa aggtctcaac |
| 241 | tttgcagaat tttcctctaa aggttcagac tttggggtaa aggtgtcaac tttggcgatg |
| 301 | ggtcttgacg gaaacaatgg tggaggggtt tggttaaacg gtggtggtgg agaaagggaa |
| 361 | gagaacgagg aaggttcatg gggaaggaat caagaagatg gttcttctca gtttaagcct |
| 421 | atgcttgaag gtgattggtt tagtagtaac caaccacatc cacaagatct tcagatgtta |
| 481 | cagaatcagc cagatttcag atactttggt ggttttcctt ttaaccctaa tgataatctt |
| 541 | cttcttcaac actctattga ttcttcttct tcttgttctc cttctcaagc ttttagtctt |
| 601 | gacccttctc agcaaaatca gttcttgtca actaacaaca acaagggttg tcttctcaat |
| 661 | gttccttctt ctgcaaaccc ttttgataat gcttttgagt ttggctctga atctggtttt |
| 721 | cttaaccaaa tccatgctcc tatttcgatg gggtttggtt ctttgacaca attggggaac |
| 781 | agggatttga gttctgttcc tgatttcttg tctgctcggt cacttcttgc gccggaaagc |
| 841 | aacaacaaca acacaatgtt gtgtggtggt ttcacagctc cgttggagtt ggaaggtttt |
| 901 | ggtagtcctg ctaatggtgg ttttgttggg aacagagcga aagttctgaa gcctttagag |
| 961 | gtgttagcat cgtctggtgc acagcctact ctgttccaga aacgtgcagc tatgcgtcag |
| 1021 | agctctggaa gcaaaatggg aaattcggag agttcgggaa tgaggaggtt tagtgatgat |
| 1081 | ggagatatgg atgagactgg gattgaggtt tctgggttga actatgagtc tgatgagata |
| 1141 | aatgagagcg gtaaagcggc tgagagtgtt cagattggag gaggaggaaa gggtaagaag |
| 1201 | aaaggtatgc ctgctaagaa tctgatggct gagaggagaa ggaggaagaa gcttaatgat |
| 1261 | aggctttata tgcttagatc agttgtcccc aagatcagca aaatggatag agcatcaata |
| 1321 | cttggagatg caattgatta tctgaaggaa cttctacaaa ggatcaatga tcttcacaat |
| 1381 | gaacttgagt caactcctcc tggatctttg cctccaactt catcaagctt ccatccgttg |
| 1441 | acacctacac cgcaaactct ttcttgtcgt gtcaaggaag agttgtgtcc ctcttcttta |
| 1501 | ccaagtccta aaggccagca agctagagtt gaggttagat taagggaagg aagagcagtg |
| 1561 | aacattcata tgttctgtgg tcgtagaccg ggtctgttgc tcgctaccat gaaagctttg |
| 1621 | gataatcttg gattggatgt tcagcaagct gtgatcagct gttttaatgg gtttgccttg |
| 1681 | gatgttttcc gcgctgagca atgccaagaa ggacaagaga tactgcctga tcaaatcaaa |
| 1741 | gcagtgcttt tcgatacagc agggtatgct ggtatgatct gatctgatcc tgacttcgag |
| 1801 | tccattaagc atctgttgaa gcagagctag aagaactaag tccctttaaa tctgcaattt |
| 1861 | tcttctcaac tttttttctt atgtcataac ttcaatctaa gcatgtaatg caattgcaaa |
| 1921 | tgagagttgt ttttaaatta agcttttgag aacttgaggt tgttgttgtt ggatacataa |
| 1981 | cttcaacctt ttattagcaa tgttaacttc catttatgtc t |

ICE1 is predicted to encode a protein of 494 amino acids, with an estimated molecular mass of 53.5 kDa as follows (SEQ ID NO: 2):

MGLDGNNGGGVWLNGGGGEREENEEGSWGRNQEDGSSQFKPMLEGDWFSSNQPHPQDLQMLQNQP
DFRYFGGFPFNPNDNLLLQHSIDSSSSCSPSQAFSLDPSQQNQFLSTNNNKGCLLNVPSSANPFDNAFEF
GSESGFLNQIHAPISMGFGSLTQLGNRDLSSVPDFLSARSLLAPESNNNNTMLCGGFTAPLELEGFGSPA
NGGFVGNRAKVLKPLEVLASSGAQPTLFQKRAAMRQSSGSKMGNSESSGMRRFSDDGDMDETGIEVS
GLNYESDEINESGKAAESVQIGGGGKGKKKGMPAKNLMAERRRRKKLNDRLYMLRSVVPKISKMDR
ASILGDAIDYLKELLQRINDLHNELESTPPGSLPPTSSSFHPLTPTPQTLSCRVKEELCPSSLPSPKGQQAR
VEVRLREGRAVNIHMFCGRRPGLLLATMKALDNLGLDVQQAVISCFNGFALDVFRAEQCQEGQEILPD
QIKAVLFDTAGYAGMI

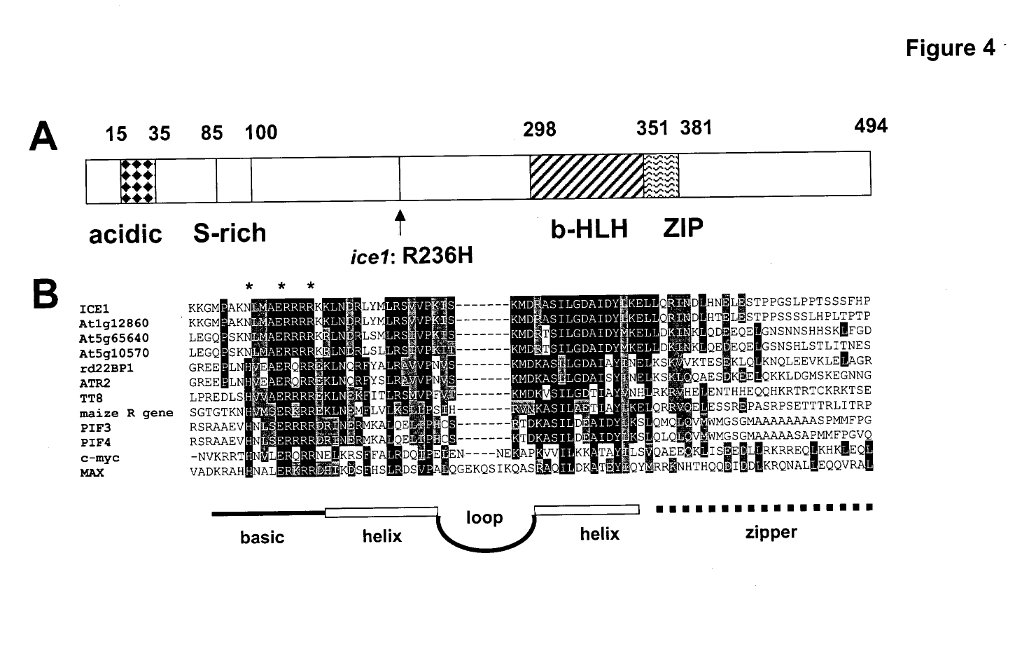

Database searches revealed that ICE1 contains a MYC-like basic helix-loop-helix (bHLH) domain at its C-terminal half (FIGS. 4A and 4B). Over the entire length of the protein, ICE1 shows amino acid sequence similarity to an unknown protein of Arabidopsis (At1g12860). The ice1 mutation changes Arg236, conserved in these two Arabidopsis proteins, to His. The bHLH domain of ICE1 shows high amino acid similarity to that of known MYC-related bHLH transcription factors (FIG. 4B). All MYC binding promoter elements contain the CA nucleotides that are contacted by a conserved glutamic acid in the bHLH zipper domain (Grandori et al., 2000). This glutamic acid residue (Glu312) is also conserved in the basic DNA binding domain of ICE1 (FIG. 4B). An acidic domain near the amino terminus characterizes the bHLH family of transcription factors and a conserved bHLH DNA binding and dimerization domain near the carboxyl terminus (Purugganan and Wessler 1994). All these features are present in ICE1 protein (FIG. 4A).

To analyze the expression pattern of ICE1 in different tissues, T2 lines of transgenic Arabidopsis plants expressing an ICE1 promoter-GUS transgene were analyzed. GUS expression was detected in roots, leaves, stem and floral parts. Semi-quantitative RT-PCR analysis also showed that ICE1 was expressed constitutively and the expression was stronger in leaves and stems than in other tissues (FIGS. 5A and 5B). RNA blot analysis showed that the ICE1 transcript was slightly up-regulated by cold, NaCl and ABA but not by dehydration (FIG. 5C).

To examine the subcellular localization of the ICE1 protein, ICE1 was fused in-frame to the C-terminal side of the green fluorescent protein (GFP) and expressed under control of the CaMV 35S promoter. Confocal imaging of GFP fluorescence in T2 transgenic plants showed that the GFP-ICE1 fusion protein is present in the nucleus under either warm (FIG. 5D) or cold temperatures.

ICE1 Binds to MYC Recognition Sites in the CBF3 Promoter

ICE1 has a basic helix-loop-helix (bHLH) domain and its amino acid sequence in the basic region is highly conserved with other bHLH proteins (FIG. 4B), and therefore may recognize promoter elements similar to the DNA-binding sites for known bHLH proteins. These proteins recognize DNA with the consensus sequence CANNTG (Meshi and Iwabuchi 1995). In the promoter region of CBF3, there are five potential MYC-recognition elements within a 1 kb region upstream of the transcription initiation site (Shinwari et al. 1998). These possible MYC-recognition sites, designated MYC-1 through MYC-5, fall into four groups because MYC-3 and MYC-5 share the same consensus sequence, CATTTG (FIG. 6A). Thus, MYC-3 was used to represent both MYC-3 and MYC-5. To determine whether ICE1 binds to these MYC-recognition sites in the CBF3 promoter, we expressed and purified His-ICE1 fusion protein from E. coli. Four DNA fragments encompassing each possible MYC-recognition site were used for interaction with His-ICE1 in an electrophoresis mobility shift assay (EMSA).

Several complexes were observed when ICE1 was incubated with any of the four DNA fragments (MYC-1 through MYC-4), indicating that ICE1 is able to bind to these sequences (FIG. 6B). The MYC-2 fragment formed one major complex with ICE1, while the other DNA fragments formed several complexes with ICE1. These complexes were abolished by the addition of increasing amounts of cold competitors with the same sequences, but not by P1 or P2, which contains a putative MYB- recognition site and a non-related sequence, respectively (FIG. 6B). This specificity of competition strengthens the hypothesis that the interaction between DNA and ICE1 requires the MYC-recognition sequences. When the MYC-2 fragment was used as a probe, the complex was most efficiently competed off by the cold MYC-2 competitor, suggesting that ICE1 has a higher affinity for the MYC-2 site than for the other sites (FIG. 6C). The complex formed by ICE1 and the MYC-2 fragment was less affected by a mutated competitor than by the wild type competitor (FIG. 6D). Together, these results show that ICE1 interacts specifically with the MYC-recognition sites in the CBF3 promoter. The ice1 mutation does not appear to affect ICE1 interaction with the CBF3 promoter, because the Arg236 to His mutant form of ICE1 was also able to bind to the MYC-2 probe (FIG. 6E).

ICE1 is a Transcriptional Activator that Positively Regulates CBF Expression

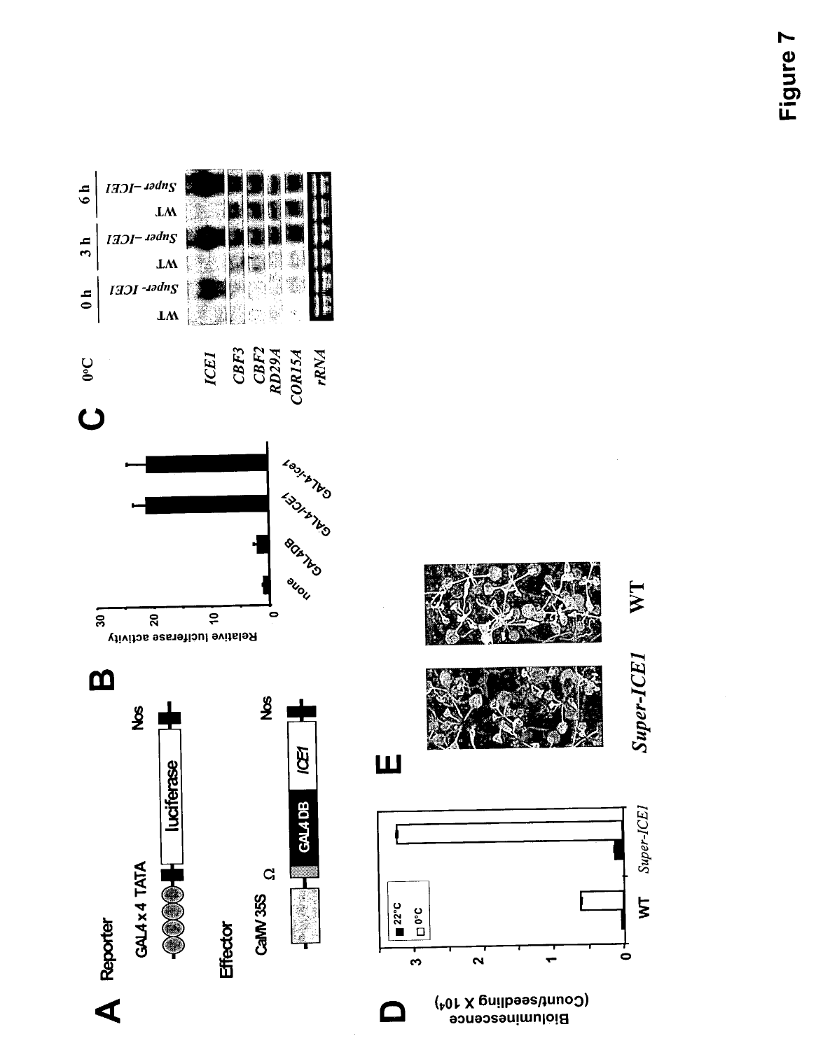

Transient expression assays were carried out to determine whether ICE1 acts as a transcriptional activator or repressor. An effector plasmid was constructed by fusing ICE1 with the DNA binding domain of the yeast GAL4 transcriptional activator (GAL4-ICE1, FIG. 7A). When the wild type GAL4-ICE1 and a GAL4-responsive reporter gene, GAL4-LUC, were delivered into Arabidopsis leaves by particle bombardment, the luciferase activity increased 20 fold relative to the control with or without an effector plasmid containing only the GAL4 DNA binding domain (FIG. 7B). The Arg236 to His mutant form of GAL4-ICE1 also activated the GAL4-responsive transcription (FIG. 7B). These results suggest that ICE1 is a transcriptional activator, and that the ice1 mutation does not affect the function of the transcriptional activation domain.

A null allele of ice1 created by T-DNA insertion does not show any phenotypes of the dominant ice1 mutant, suggesting that there is functional redundancy in the ICE1 gene family. We overexpressed ICE1 in wild type Arabidopsis plants by using the strong constitutive super promoter. None of the overexpression lines showed any ice1 mutant phenotypes. RNA blot analysis showed that ICE1 -overexpression did not activate CBF3 expression at warm temperatures. However, ICE1 -overexpression enhanced the expression of the endogenous CBF3 gene as well as the CBF3-LUC reporter gene in the cold (FIGS. 7C and 7D). Cold-induction of CBF2, RD29A and COR15A was also enhanced in the Super-25 ICE1 transgenic plants (FIG. 7C). When the Super-ICE1transgenic plants and wild type control plants in the same agar plates were cold acclimated at 4° C. for 5 days and then subjected to freezing treatment at −8° C. for 4 hours, the ICE1 overexpression transgenic seedlings showed a higher survival rate (75.9±6.5%) than that of control plants (37.2±12.6%) (FIG. 7E). The ICE1 overexpression transgenic plants did not exhibit obvious growth or developmental abnormalities. These results suggest that ICE1 is a positive regulator of CBF3, and that the dominant nature of ice1 is likely caused by a dominant negative effect of the mutation.

Numerous modifications and variations on the present invention are possible in light of the above teachings. It is, therefore, to be understood that within the scope of the accompanying claims, the invention may be practiced otherwise than as specifically described herein.

### REFERENCES

Browse, J. and Xin, Z. 2001. Temperature sensing and cold acclimation. Curr. Opin. Plant Biol. 4: 241-246.

Chinnusamy, V., Stevenson, B., Lee, B. -h., and Zhu, J. -K. 2002. Screening for gene regulation mutants by bioluminescence imaging. Science 's STKE http://stke.sciencemag.org/cgi/content/full/sigtrans;2002/140/p110 (Jul. 9, 2002).

Fowler, S., and Thomashow, M. F. 2002. Arabidopsis transcriptome profiling indicates that multiple regulatory pathways are activated during cold acclimation in addition to the CBF cold response pathway. Plant Cell 14: 1675-1690.

Gilmour, S. J., Artus, N. N., and Thomashow, M. F. 1992. cDNA sequence analysis and expression of two cold regulated genes of Arabidopsis thaliana. Plant Mol. Biol. 18: 13-32.

Gilmour, S. J., Zarka, D. G., Stockinger, E. J., Salazar, M. P., Houghton, J. M., and Thomashow, M. F. 1998. Low temperature regulation of the Arabidopsis CBF family of AP2 transcriptional activators as an early step in cold-induced COR gene expression. Plant J. 16: 433-442.

Gong, Z., Lee, H., Xiong, L., Jagendorf, A., Stevenson, B., and Zhu, J.-K. 2002. RNA helicase-like protein as an early regulator of transcription factors for plant chilling and freezing tolerance. Proc. Natl. Acad. Sci. USA 99: 11507-11512.

Grandori, C., Cowley, S. M., James, L. P. and Eisenman, R. N. 2000. The MYC/MAX/MAD network and the transcriptional control of cell behavior. Annu. Rev. Cell. Dev. Biol. 16: 653-699.

Guo, Y., Xiong, L., Ishitani, M. and Zhu, J. -K. 2002. An Arabidopsis mutation in translation elongation factor 2 causes superinduction of CBF/DREB 1 transcription factor genes but blocks the induction of their downstream targets under low temperature. Proc. Natl. Acad. Sci. USA. 99: 7786-7791.

Guy, C. L. 1990. Cold acclimation and freezing stress tolerance: role of protein metabolism. Annu. Rev. Plant Physiol. Plant Mol. Biol. 41: 187-223.

Hao, D., Ohme-Takagi, M., and Sarai, A. 1998. Unique mode of GCC box recognition by the DNA-binding domain of ethylene-responsive element-binding factor (ERF domain) in plant. J. Biol. Chem. 273: 26857-26861.

Hsieh, T. H., Lee, J. T., Yang, P. T., Chiu, L. H., Charng, Y. Y., Wang, Y. C., and Chan, M. T. 2002. Heterology expression of the Arabidopsis C-repeat/dehydration response element binding factor 1 gene confers elevated tolerance to chilling and oxidative stresses in transgenic tomato. Plant Physiol. 129: 1086-1094.

Hughes, M. and Dunn, M. 1996. The molecular biology of plant acclimation to low temperature. J. Exp. Bot. 47: 291-305.

Ishitani, M., Xiong, L., Lee, H., Stevenson, B., and Zhu, J. K. 1998. HOS1, a genetic locus involved in cold-responsive gene expression in Arabidopsis. Plant Cell 10: 1151-1161.

Ishitani, M., Xiong, L., Stevenson, B., and Zhu, J. K. 1997. Genetic analysis of osmotic and cold stress signal transduction in Arabidopsis: interactions and convergence of abscisic acid-dependent and abscisic acid-independent pathways. Plant Cell 9: 1935-1949.

Jaglo-Ottosen, K. R., Gilmour, S. J., Zarka, D. G., Schabenberger, O., and Thomashow, M. F. 1998. Arabidopsis CBF1 overexpression induces cor genes and enhances freezing tolerance. Science 280: 104-106.

Knight, H., Veale, E. L., Warren, G. J., and Knight, M. R. 1999. The sfr6 mutation in Arabidopsis suppresses low-temperature induction of genes dependent on the CRT/DRE sequence motif. Plant Cell 11: 875-886.

Kurkela, S., and Franck, M. 1990. Cloning and characterization of a cold- and ABA-inducible Arabidopsis gene. Plant Mol. Biol. 15: 137-144.

Lee, H., Guo, Y., Ohta, M., Xiong, L., Stevenson, B., and Zhu, J.-K. 2002. LOS2, a genetic locus required for cold-responsive gene transcription encodes a bifunctional enolase. EMBO. J. 21: 2692-2702.

Lee, H., Xiong, L., Gong, Z. , Ishitani, M., Stevenson, B., and Zhu, J. K. 2001. The Arabidopsis HOS1 gene negatively regulates cold signal transduction and encodes a RING finger protein that displays cold-regulated nucleo-cytoplasmic partitioning. Genes Dev. 15: 912-924.

Li, X., Gong, Z., Koiwa, H., Niu, X., Espartero, J., Zhu, X., Veronese, P., Ruggiero, B., Bressan, R., Weller, S. C., and Hasegawa, P. M. 2001. Bar-expressing peppermint (Mentha×Piperita L. var. Black Mitcham) plants are highly resistant to the glufosinate herbicide Liberty. Mol. Breed. 8: 109-118.

Lin, C., and Thomashow, M. F. 1992. DNA sequence analysis of a complementary DNA for cold-regulated Arabidopsis gene cor15 and characterization of the COR15 polypeptide. Plant Physiol. 99: 519-525.

Liu, J., and Zhu, J. -K. 1997. Proline accumulation and salt-stress-induced gene expression in a salt-hypersensitive mutant of Arabidopsis. Plant Physiol. 114: 591-596.

Liu, Q., Sakuma, Y., Abe, H., Kasuga, M., Miura, S., Yamaguchi-Shinozaki, K., and Shinozaki, K. 1998. Two transcription factors, DREB1 and DREB2, with an EREBP/AP2 DNA binding domain, separate two cellular signal transduction pathways in drought- and low temperature-responsive gene expression, respectively, in Arabidopsis. Plant Cell 10: 1391-1406.

Liu, L., White, M. J., and MacRae, T. H. 1999. Transcription factors and their genes in their genes in higher plants: Functional domains, evolution and regulation. Eur. J. Biochem. 262: 247-257.

Meshi, T., and Iwabuchi, M. 1995. Plant transcription factors. Plant Cell. Physiol. 36: 1405-1420.

Mohapatra, S. S., Wolfraim, L., Poole, R. J., and Dhindsa, R. S. 1989. Molecular cloning and relationship to freezing tolerance of cold-acclimation-specific genes of alfalfa. Plant Physiol. 89: 375-380.

Ohta, M., Ohme-Takagi, M., and Shinshi, H. 2000. Three ethylene-responsive transcription factors in tobacco with distinct transactivation functions. Plant J. 22: 29-38.

Ohta, M., Matsui, K., Hiratsu, K., Shinshi, H., and Ohme-Takagi, M. 2001. Repression Domains of Class II ERF transcriptional repressors share an essential motif for active repression. Plant Cell 13: 1959-1968.

Purugganan, M. D., and Wessler, S. R. 1994. Molecular evolution of the plant R regulatory gene family. Genetics 138: 849-854.

Shinwari, Z. K., Nakashima, K., Miura, S., Kasuga, M., Seki, M., Yamaguchi-Shinozaki, K., and Shinozaki, K. 1998. An Arabidopsis gene family encoding DRE/CRT binding proteins involved in low-temperature-responsive gene expression. Biochem. Biophys. Res. Commun. 250: 161-170.

Spelt, C., Quattrocchio, F., Mol, J. N. M., and Koes, R. 2000. Anthocyanin1 of petunia encodes a basic-helix-loop-helix protein that directly activates transcription of structural anthocyanin genes. Plant Cell 12: 1619-1631.

Stockinger, E. J. Gilmour, S. J., and Thomashow, M. F. 1997. Arabidopsis thaliana CBF1 encodes an AP2 domain-containing transcription activator that binds to the C-repeat/DRE, a cis-acting DNA regulatory element that stimulates transcription in response to low temperature and water deficit. Proc. Natl. Acad. Sci. USA 94: 1035-1040.

Tahtiharju, S. and Palva, T. 2001. Antisense inhibition of protein phosphatase 2C accelerates cold acclimation in Arabidopsis thaliana. Plant J. 26: 461-470.

Thomashow, M. F. 1999. Plant cold acclimation, freezing tolerance genes and regulatory mechanisms. Annu. Rev. Plant Physiol. Plant Mol. Biol. 50: 571-599.

Walker, A. R., Davison, P. A., Bolognesi-Winfield, A. C., James, C. M., Srinivasan, N., Blundell, T. L., Esch, J. J., Marks, M. D., and Gray, J. C. 1999. The TRANSPARENT TESTA GLABRA1 locus, which regulates trichome differentiation and anthocyanin biosynthesis in Arabidopsis, encodes a WD40-repeat protein. Plant Cell 11: 1337-1349.

Xin, Z., and J. Browse 1998. eskimo1 mutants of Arabidopsis are constitutively freezing-tolerant. Proc. Natl. Acad. Sci. USA 95: 7799-7804.

Yamaguchi-Shinozaki, K., and Shinozaki, K. 1994. A novel cis-acting element in an Arabidopsis gene is involved in responsiveness to drought, low-temperature, or high-salt stress. Plant Cell 6: 251-264.

What we claim is:

1. An isolated polynucleotide which encodes a protein comprising the amino acid sequence of SEQ ID NO:2.

2. The isolated polynucleotide of claim 1, wherein said protein has ICE1 transcriptional activator activity.

3. An isolated polynucleotide, which comprises the polynucleotide of SEQ ID NO:1.

4. An isolated polynucleotide which is complimentary to the polynucleotide of claim 3.

5. An isolated polynucleotide which is at least 10% identical to the polynucleotide of claim 3.

6. An isolated polynucleotide which is at least 80% identical to the polynucleotide of claim 3.

7. An isolated polynucleotide which is at least 90% identical to the polynucleotide of claim 3.

8. An isolated polynucleotide which hybridizes under stringent conditions to the polynucleotide of claim 3; wherein said stringent conditions comprise washing in 5×SSC at a temperature from 50 to 68° C.

9. The isolated polynucleotide of claim 3, which encodes a protein having ICE1 transcriptional activator activity.

10. A vector comprising the isolated polynucleotide of claim 1.

11. A vector comprising the isolated polynucleotide of claim 3.

12. A host cell comprising the isolated polynucleotide of claim 1.

13. A host cell comprising the isolated polynucleotide of claim 3.

14. A plant cell comprising the isolated polynucleotide of claim 1.

15. A plant cell comprising the isolated polynucleotide of claim 3.

16. A transgenic plant comprising the isolated polynucleotide sequence of claim 1.

17. A transgenic plant comprising the isolated polynucleotide sequence of claim 3.

18. The transgenic plant of claim 16, wherein said plant is Arabidopsis thaliania.

19. The transgenic plant of claim 17, wherein said plaint is Arabidopsis thaliania.

20. The transgenic plant of claim 16, wherein said plant is selected from the group consisting of wheat, corn, peanut cotton, oat, and soybean plant.

21. The transgenic plant of claim 16, wherein the isolated polynucleotide is operably linked to an inducible promoter.

22. The transgenic plant of claim 17, wherein the isolated polynucleotide is operably linked to an inducible promoter.

23. A process for screening for polynucleotides which encode a protein ICE1 transcriptional activator activity comprising hybridizing the isolated polynucleotide of claim 1 to the polynucleotide to be screened; expressing the polynucleotide to produce a protein; and detecting the presence or absence of ICE1 transcriptional activator activity in said protein.

24. A process for screening for polynucleotides which encode a protein having ICE1 transcriptional activator activity comprising hybridizing the isolated polynucleotide of claim 3 to the polynucleotide to be screened; expressing the polynucleotide to produce a protein; and detecting the presence or absence of ICE1 transcriptional activator activity in said protein.

25. A process for screening for polynucleotides which encode a protein having ICE1 transcriptional activator activity comprising hybridizing the isolated polynucleotide of claim 8 to the polynucleotide to be screened; expressing the polynucleotide to produce a protein; and detecting the presence or absence of ICE1 transcriptional activator activity in said protein.

26. A method for detecting a nucleic acid with at least 70% homology to nucleotide of claim 1, comprising contacting a nucleic acid sample with a probe or primer comprising at least 15 consecutive nucleotides of the nucleotide sequence of claim 1, or at least 15 consecutive nucleotides of the complement thereof.

27. A method for producing a nucleic acid with at least 70% homology to nucleotide of claim 1, comprising contacting a nucleic acid sample with a primer comprising at least 15 consecutive nucleotides of the nucleotide sequence of claim 1, or at least 15 consecutive nucleotides of the complement thereof.

28. A method for the polynucleotide of claim 3, comprising contacting a nucleic acid sample with a probe or primer comprising at least 15 consecutive nucleotides of the nucleotide sequence of claim 3, or at least 15 consecutive nucleotides of the complement thereof.

29. A method for producing the polynucleotide of claim 3, comprising contacting a nucleic acid sample with a primer comprising at least 15 consecutive nucleotides of the nucleotide sequence of claim 3, or at least 15 consecutive nucleotides of the complement thereof.

30. A method for making ICE1 protein, comprising culturing the host cell of claim 12 for a time and under conditions suitable for expression of ICE1, and collecting the ICE1 protein.

31. A method for making ICE1, comprising culturing the host cell of claim 13 for a time and under conditions suitable for expression of ICE1, and collecting the ICE1 protein.

32. A method of making a transgenic plant comprising introducing the polynucleotide of claim 1 into the plant.

33. A method of making a transgenic plant comprising introducing the polynucleotide of claim 1 into the plant.

34. A method of increasing cold acclimation of a plant in need thereof, comprising introducing the polynucleotide of claim 1 into said plant.

35. A method of increasing cold acclimation of a plant in need thereof, comprising introducing the polynucleotide of claim 3 into said plant.

36. A method of increasing cold acclimation of a plant in need thereof, comprising enhancing the expression of the ice1 gene in said plant.

37. An isolated polypeptide comprising the amino acid sequence in SEQ ID NO:2.

38. The isolated polypeptide of claim 37 which has ICE1 transcriptional activator activity.

39. An isolated polypeptide which is at least 70% identical to the isolated polypeptide of claim 37 and which has ICE1 transcriptional activator activity.

40. An isolated polypeptide which is at least 80% identical to the isolated polypeptide of claim 37 and which has ICE1 transcriptional activator activity.

41. An isolated polypeptide which is at least 90% identical to the isolated polypeptide of claim 37 and which has ICE1 transcriptional activator activity.

42. An isolated polypeptide which is at least 95% identical to the isolated polypeptide of claim 37 and which has ICE1 transcriptional activator activity.

43. A method of increasing cold acclimation in a plant, comprising overexpressing an ICE1 transcriptional activator in the plant.

44. The method of claim 43, wherein the ICE1 transcriptional activator has the amino acid sequence of SEQ ID NO: 2.

45. The method of claim 43, wherein the ICE1 transcriptional activator is encoded by a nucleic acid having the sequence of SEQ ID NO: 1.

46. The method of claim 43, wherein the ICE1 transcriptional activator is encoded by a nucleic acid which has a sequence which is at least 70% identical to SEQ ID NO: 1.

47. The method of claim 43, wherein the ICE1 transcriptional activator is encoded by a nucleic acid which has a sequence which is at least 90% identical to SEQ ID NO: 1.

48. The method of claim 43, wherein the ICE1 transcriptional activator is encoded by a nucleic acid which hybridizes under stringent conditions to the complement of SEQ ID NO: 1, wherein said stringent conditions comprise washing in 5×SSC at a temperature of form 50 to 68° C.

49. The method of claim 43, wherein the amino acid sequence of the ICE1 transcriptional activator has a homology of at least 80% with SEQ ID NO: 2.

50. The method of claim 43, wherein the amino acid sequence of the ICE1 transcriptional activator has a homology of at least 90% with SEQ ID NO: 2.

51. The method of claim 43, wherein the plant is Arabidopsis thalania.

52. The method of claim 43, wherein the plant is selected from the group consisting of wheat, corn, peanut cotton, oat, and soybean.

53. The method of claim 43, wherein the plants have an increased expression of one or more additional transcription factors selected from the group consisting of a CBF transcription factor and a DREB 1 transcription factor.

54. The method of claim 43, wherein the plants have an increased expression of one or more cold-responsive genes.

55. The method of claim 54, wherein the cold responsive genes encode a protein selected from the group consisting of an enzyme involved in respiration of carbohydrates, an enzyme involved in metabolism of carbohydrates, an enzyme involved in respiration of lipids, an enzyme involved in metabolism of lipids, an enzyme involved in respiration of phenylpropanoids, an enzyme involved in metabolism of phenylpropanoids, an enzyme involved in respiration of antioxidants, an enzyme involved in metabolism of antioxidants, a molecular chaperone, an antifreeze protein, and a protein involved in tolerance to the dehydration caused by freezing.

56. The method of claim 43, wherein the plant is transformed with a vector encoding the ICE1 transcriptional activator.

57. The method of claim 56, wherein the ICE1 transcriptional activator has the amino acid sequence of SEQ ID NO: 2.

58. The method of claim 56, wherein ICE1 transcriptional activator is encoded by a nucleic acid having the sequence of SEQ ID NO: 1.

59. The method of claim 56, wherein the ICE1 transcriptional activator is encoded by a nucleic acid which has a sequence which is at least 70% identical to SEQ ID NO: 1.

60. The method of claim 56, wherein the ICE1 transcriptional activator is encoded by a nucleic acid which has a sequence which is at least 90% identical to SEQ ID NO: 1.

61. The method of claim 56, wherein the ICE1 transcriptional activator is encoded by a nucleic acid which hybridizes under stringent conditions to the complement of SEQ ID NO: 1, wherein said stringent conditions comprise washing in 5×SSC at a temperature of form 50 to 68° C.

62. The method of claim 56, wherein the amino acid sequence of ICE1 transcriptional activator has a homology of at least 80% with SEQ ID NO: 2.

63. The method of claim 56, wherein the amino acid sequence of the ICE1 transcriptional activator has a homology of at least 90% with SEQ ID NO: 2.

64. The method of claim 56, wherein the plant is Arabidopsis thalania.

65. The method of claim 56, wherein the plant is selected from the group consisting of wheat, corn, peanut cotton, oat, and soybean.

66. The method of claim 56, wherein the plants have an increased expression of one or more additional transcription factors selected from the group consisting of a CBF transcription factor and a DREB1 transcription factor.

67. The method of claim 56, wherein the plants have an increased expression of one or more cold-responsive genes.

68. The method of claim 67, wherein the cold responsive genes encode a protein selected from the group consisting of an enzyme involved in respiration of carbohydrates, an enzyme involved in metabolism of carbohydrates, an enzyme involved in respiration of lipids, an enzyme involved in metabolism of lipids, an enzyme involved in respiration of phenylpropanoids, an enzyme involved in metabolism of phenylpropanoids, an enzyme involved in respiration of antioxidants, an enzyme involved in metabolism of antioxidants, a molecular chaperone, an antifreeze protein, and a protein involved in tolerance to the dehydration caused by freezing.

69. A method of enhancing expression of one or more cold-responsive genes in a plant cell, comprising transforming the plant with a vector which encodes an ICE1 transcriptional activator.

70. The method of claim 69, wherein the plants have increased cold acclimation.

71. The method of claim 69, wherein the ICE1 transcriptional activator has the amino acid sequence of SEQ ID NO: 2.

72. The method of claim 69, wherein the ICE1 transcriptional activator is encoded by a nucleic acid having the sequence of SEQ ID NO: 1.

73. The method of claim 69, wherein the ICE1 transcriptional activator is encoded by a nucleic acid which has a sequence which is at least 70% identical to SEQ ID NO: 1.

74. The method of claim 69, wherein the ICE1 transcriptional activator is encoded by a nucleic acid which has a sequence which is at least 90% identical to SEQ ID NO: 1.

75. The method of claim 69, wherein the ICE1 transcriptional activator is encoded by a nucleic acid which hybridizes under stringent conditions to the complement of SEQ ID NO: 1, wherein said stringent conditions comprise washing in 5×SSC at a temperature of form 50 to 68° C.

76. The method of claim 69, wherein the amino acid sequence of the ICE1 transcriptional activator has a homology of at least 80% with SEQ ID NO: 2.

77. The method of claim 69, wherein the amino acid sequence of the ICE1 transcriptional activator has a homology of at least 90% with SEQ ID NO: 2.

78. The method of claim 69, wherein the plant cell is Arabidopsis thalania.

79. The method of claim 69, wherein the plant cell is selected from the group consisting of wheat, corn, peanut cotton, oat, and soybean.

80. An expression cassette comprising a promoter functional in a plant cell operably linked to an isolated nucleic acid encoding an ICE1 protein of SEQ ID NO: 2, wherein enhanced expression of the protein in a plant cell imparts increased cold acclimation to said plant cell.

81. The expression cassette of claim 80, wherein the promoter is selected from the group consisting of a viral coat protein promoter, a tissue-specific promoter, a monocot promoter, a ubiquitin promoter, a stress inducible promoter, a CaMV 35S promoter, a CaMV 19S promoter, an actin promoter, a cab promoter, a sucrose synthase promoter, a tubulin promoter, a napin R gene complex promoter, a tomato E8 promoter, a patatin promoter, a mannopine synthase promoter, a soybean seed protein glycinin promoter, a soybean vegetative storage protein promoter, a bacteriophage SP6 promoter, a bacteriophage T3 promoter, a bacteriophage T7 promoter, a Ptac promoter, a root-cell promoter, an ABA-inducible promoter and a turgor-inducible promoter.
